(19) United States
(12) Patent Application Publication
(10) Pub. No.: US 2025/0143924 A1
(43) Pub. Date: May 8, 2025

(54) TRANSVITREAL SUBRETINAL INJECTOR

(71) Applicant: Genentech, Inc., South San Francisco, CA (US)

(72) Inventors: Chase Barrett Wooley, New Albany, IN (US); Ben Morris, Jeffersonville, IN (US); Thomas E. Meyer, Philadelphia, PA (US); Sergio Camacho, King of Prussia, PA (US); Mark James Hedgeland, Norristown, PA (US); Peter James Meinz, Minneapolis, MN (US)

(21) Appl. No.: 19/016,576

(22) Filed: Jan. 10, 2025

Related U.S. Application Data
(63) Continuation of application No. PCT/IB2023/000397, filed on Jul. 10, 2023.
(60) Provisional application No. 63/388,150, filed on Jul. 11, 2022.

(51) Int. Cl. A61F 9/007

(52) U.S. Cl. CPC A61F 9/00736

(57) ABSTRACT

An apparatus includes a body and an outer cannula extending distally from the body along a longitudinal axis and fixedly secured to the body. The outer cannula includes a proximal portion having a first cross-dimension and a distal portion having a second cross-dimension smaller than the first cross-dimension. The distal portion is configured to guide the cylindrical proximal portion into a trocar port. The apparatus also includes an inner cannula slidably disposed in the outer cannula for translating longitudinally relative to the outer cannula. The inner cannula includes an open proximal end, an open distal end configured to pierce a retina of the eye, and a lumen extending between the open proximal end and the open distal end. The lumen is configured to communicate a fluid from the open proximal end to the open distal end for delivering the fluid into a subretinal region of the eye.

### PRIORITY

This application claims the benefit of U.S. Pat. App. No. 63/388,150, entitled “Transvitreal Subretinal Injector,” filed Jul. 11, 2022, the disclosure of which is incorporated by reference herein.

### BACKGROUND

The human eye comprises several layers. The white outer layer is the sclera, which surrounds the choroid layer. The retina is interior to the choroid layer. The sclera contains collagen and elastic fiber, providing protection to the choroid and retina. The choroid layer includes vasculature providing oxygen and nourishment to the retina. The retina comprises light sensitive tissue, including rods and cones. The macula is located at the center of the retina at the back of the eye, generally centered on an axis passing through the centers of the lens and cornea of the eye (i.e., the optic axis). The macula provides central vision, particularly through cone cells.

Macular degeneration is a medical condition that affects the macula, such that people suffering from macular degeneration may experience lost or degraded central vision while retaining some degree of peripheral vision. Macular degeneration may be caused by various factors such as age (also known as “AMD”) and genetics. Macular degeneration may occur in a “dry” (nonexudative) form, where cellular debris known as drusen accumulates between the retina and the choroid, resulting in an area of geographic atrophy. Macular degeneration may also occur in a “wet” (exudative) form, where blood vessels grow up from the choroid behind the retina. Even though people having macular degeneration may retain some degree of peripheral vision, the loss of central vision may have a significant negative impact on the quality of life. Moreover, the quality of the remaining peripheral vision may be degraded and in some cases may disappear as well. It may therefore be desirable to provide treatment for macular degeneration in order to prevent or reverse the loss of vision caused by macular degeneration. In some cases it may be desirable to provide such treatment in a highly localized fashion, such as by delivering a therapeutic substance in the subretinal layer (under the neurosensory layer of the retina and above the retinal pigment epithelium) directly adjacent to the area of geographic atrophy, near the macula. However, since the macula is at the back of the eye and underneath the delicate layer of the retina, it may be difficult to access the macula in a practical fashion.

While a variety of surgical methods and instruments have been made and used to treat an eye, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an example of an instrument for delivery of a therapeutic agent to a subretinal space;

FIG. 2A depicts a side elevational view of the instrument of FIG. 1 with the retinal penetrating element in a retracted position;

FIG. 2B depicts a side elevational view of the instrument of FIG. 1 with the retinal penetrating element in an extended position;

FIG. 3 depicts a top plan view of an eye of a patient, with surrounding structures of the eye immobilized and three ports installed;

FIG. 4A depicts a cross-sectional view of the eye of FIG. 3, the cross-section taken along line 4A-4A of FIG. 3;

FIG. 4B depicts a cross-sectional view of the eye of FIG. 3, the cross-section taken along line 4B-4B of FIG. 3, showing an outer cannula of the instrument of FIG. 1 inserted into a trocar port;

FIG. 4C depicts a cross-sectional view of the eye of FIG. 3, the cross-section taken along line 4B-4B of FIG. 3, showing the retinal penetrating element of the instrument of FIG. 1 piercing the retina of the eye to define a subretinal entry point;

FIG. 4D depicts a cross-sectional view of the eye of FIG. 3, the cross-section taken along line 4B-4B of FIG. 3, showing bleb fluid injected into the subretinal space of the eye via the retinal penetrating element of the instrument of FIG. 1 to form a leading bleb;

FIG. 4E depicts a cross-sectional view of the eye of FIG. 3, the cross-section taken along line 4B-4B of FIG. 3, showing advancement of the retinal penetrating element of the instrument of FIG. 1 between the leading bleb and the choroid of the eye;

FIG. 4F depicts a cross-sectional view of the eye of FIG. 3, the cross-section taken along line 4B-4B of FIG. 3, showing advancement of the retinal penetrating element of the instrument of FIG. 1 past the leading bleb into the space between the retina and the choroid, and further showing therapeutic fluid injected into the subretinal space via the retinal penetrating element;

FIG. 4G depicts a cross-sectional view of the eye of FIG. 3, the cross-section taken along line 4B-4B of FIG. 3, showing retraction of the retinal penetrating element of the instrument of FIG. 1 toward the subretinal entry point;

FIG. 4H depicts a cross-sectional view of the eye of FIG. 3, the cross-section taken along line 4B-4B of FIG. 3, showing aspiration of the leading bleb via the retinal penetrating element of the instrument of FIG. 1;

FIG. 4I depicts a cross-sectional view of the eye of FIG. 3, the cross-section taken along line 4B-4B of FIG. 3, showing removal of the instrument of FIG. 1 from the trocar port;

FIG. 4J depicts a cross-sectional view of the eye of FIG. 3, the cross-section taken along line 4B-4B of FIG. 3, showing sealing of the subretinal entry point;

FIG. 5 depicts an exploded perspective view of an example of a system for delivery of a therapeutic agent to a subretinal space, the system including a fluid injection instrument, a fluid source assembly, and a pressure control delivery assembly;

FIG. 6 depicts an exploded perspective view of the fluid injection instrument of FIG. 5;

FIG. 7 depicts an exploded perspective view of a portion of a fluid supply assembly and a portion of an actuation assembly of the fluid injection instrument of FIG. 5;

FIG. 8 depicts a perspective view of a portion of the actuation assembly of the fluid injection instrument of FIG. 5;

FIG. 9A depicts a side elevational view of the fluid injection instrument of FIG. 5 with the retinal penetrating element in a retracted position;

FIG. 9B depicts a side elevational view of the fluid injection instrument of FIG. 5 with the retinal penetrating element in an extended position;

FIG. 10A depicts a cross-sectional view of the fluid injection instrument of FIG. 5 with the retinal penetrating element in the retracted position;

FIG. 10B depicts a cross-sectional view of the fluid injection instrument of FIG. 5 with the retinal penetrating element in the extended position;

FIG. 11A depicts a cross-sectional view of a distal portion of the fluid injection instrument of FIG. 5 with the retinal penetrating element in the retracted position;

FIG. 11B depicts a cross-sectional view of a distal portion of the fluid injection instrument of FIG. 5 with the retinal penetrating element in the extended position;

FIG. 12A depicts a cross-sectional view of a proximal portion of the fluid injection instrument of FIG. 5 with the retinal penetrating element in the retracted position;

FIG. 12B depicts a cross-sectional view of a proximal portion of the fluid injection instrument of FIG. 5 with the retinal penetrating element in the extended position;

FIG. 12C depicts a cross-sectional view of a proximal portion of the fluid injection instrument of FIG. 5 with the retinal penetrating element in the extended position, and with a first valve of the fluid supply assembly in an open state for supplying a first fluid to the retinal penetrating element, and with a second valve of the fluid supply assembly in a closed state;

FIG. 12D depicts a cross-sectional view of a proximal portion of the fluid injection instrument of FIG. 5 with the retinal penetrating element in the extended position, with the first valve of the fluid supply assembly in a closed state, and with the second valve of the fluid supply assembly in an open state for supplying a second fluid to the retinal penetrating element;

FIG. 13 depicts an exploded perspective view of the fluid source assembly of FIG. 5;

FIG. 14 depicts an exploded perspective view of the pressure control delivery assembly of FIG. 5;

FIG. 15A depicts a top plan view of a fluid selection device of the pressure control delivery assembly of FIG. 5, with a flow selector of the fluid selection device in a first open state;

FIG. 15B depicts a top plan view of the fluid selection device of the pressure control delivery assembly of FIG. 5, with the flow selector of the fluid selection device in a second open state;

FIG. 16 depicts an exploded perspective view of another example of a system for delivery of a therapeutic agent to a subretinal space, the system including a fluid injection instrument, a fluid source assembly, and a pressure control delivery assembly;

FIG. 17 depicts an exploded perspective view of the fluid injection instrument of FIGS. 16;

FIG. 18 depicts a perspective view of a portion of an actuation assembly of the fluid injection instrument of FIG. 16;

FIG. 19 depicts a cross-sectional view of the fluid injection instrument of FIG. 16;

FIG. 20 depicts an exploded perspective view of the fluid source assembly of FIG. 16;

FIG. 21 depicts a top plan view of the fluid source assembly of FIG. 16;

FIG. 22 depicts a cross-sectional view of the fluid source assembly of FIG. 16;

FIG. 23 depicts a rear elevational view of the fluid source assembly of FIG. 16; and

FIG. 24 depicts an exploded perspective view of the pressure control delivery assembly of FIG. 16.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

I. Example of Instrument for Delivery of Therapeutic Agent from Trans-Retinal Approach

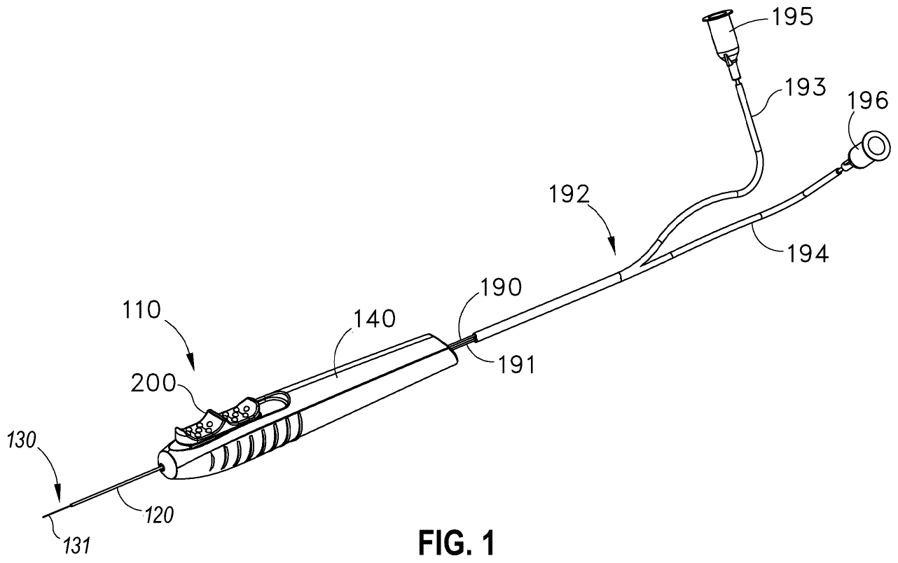

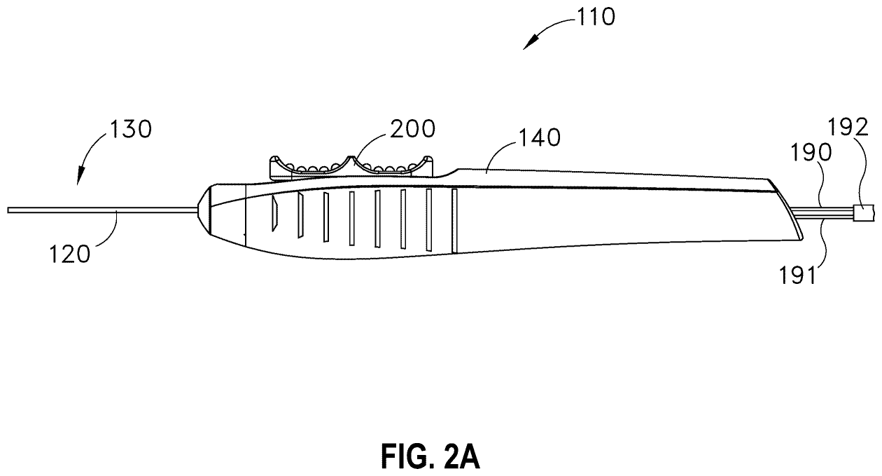

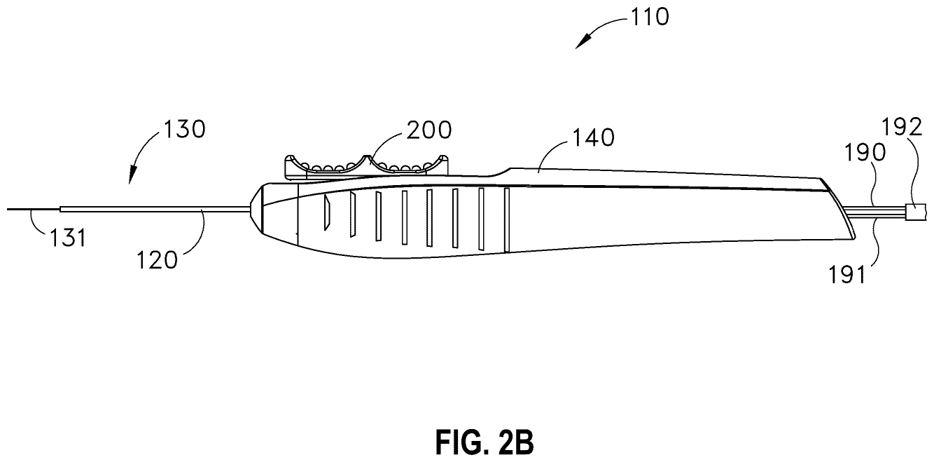

FIGS. 1-2B show an example of an instrument (110) that may be used to deliver bleb fluid (340) and therapeutic agent (341) to the subretinal space from a transvitreal, trans-retinal approach via a single lumen in a single tip. As best seen in FIG. 1, instrument (110) of this example comprises a pair of luer fittings (195, 196), a fluid supply guide (192), supply tubes (190, 191) fluidly connected to luer fittings (195, 196) respectively, a body (140), a slider (200), and a fluid delivery assembly (130). Luer fittings (195, 196) may connect to two different sources of fluid, such as leading bleb (340) fluid and therapeutic fluid (341). Luer fittings (195, 196) connect to tubing lines (193, 194) respectively, which terminate into fluid supply guide (192). While conventional luer fittings (195, 196) are used in the present example, any other suitable kind of fittings may be used.

Fluid supply guide (192) brings together tubing lines (193, 194) into a fixed, parallel relationship. Supply tubes (190, 191) exit fluid supply guide (192) and enter body (140) as will be described in greater detail below. It should be understood that fluid supply guide (192) provides a dedicated path for fluid communication from tubing line (193) to supply tube (190); and from tubing line (194) to supply tube (191). Fluid supply guide keeps the path that is associated with tubing line (193) and supply tube (190) isolated from the path that is associated with tubing line (194) and supply tube (191). The fluid sources that are coupled with luer fittings (195, 196) may be pressurized either manually (e.g., such as with a syringe), or automatically (e.g., with a pump). The operator may then choose which fluid source to pressurize to selectively pump fluids (340, 341) to corresponding supply tubes (190, 191).

Fluid delivery assembly (130) comprises an outer cannula (120) and an inner cannula (131). Outer cannula (120) is fixed relative to body (140). Outer cannula (120) is sized and configured to pass through a trocar port (318) that is inserted into the eye (301) of a patient. Inner cannula (131) is longitudinally slidable relative to outer cannula (120) and body (140). In particular, inner cannula (131) is longitudinally driven by a slider (200).

As best seen in FIGS. 2A-2B, slider (200) is slidably coupled with body (140). Slider (200) is capable of translating from a proximal position, as shown in FIG. 2A, to a distal position, as shown in FIG. 2B. Translation of slider (200) translates inner cannula (131) relative to body (140) and relative to outer cannula (120). In some versions, slider (200) is operable to slide inner cannula (131) along a longitudinal distance of up to approximately 10 mm. Alternatively, any other suitable distances may be provided. Inner cannula (131) is in fluid communication with both supply tubes (190, 191).

In the present example, the distal end of inner cannula (131) is flush cut, such that the distal edge of inner cannula extends along a plane that is perpendicular to the longitudinal axis of inner cannula (131). In other words, the distal end of inner cannula (131) is not sharp in the present example. Nevertheless, due to the small diameter of inner cannula (131), the column strength of the material forming inner cannula (131), and the fragility of the retina (308), the distal end of inner cannula (131) is capable of penetrating the retina (308) as described below, despite the blunt configuration of the distal end of inner cannula (131). In some other versions, the distal end of inner cannula (131) is sharp or has some other configuration.

Inner cannula (131) is thus capable of passing through the retina (308) to deliver fluid from either supply tube (190, 191) to the subretinal space. An example of a method of using instrument (110) in this fashion is described in greater detail below. Other suitable ways in which instrument (110) may be varied and used will be apparent to those skilled in the art in view of the teachings herein. By way of example only, instrument (110) may be configured and operable in accordance with at least some of the teachings of U.S. Pat. No. 10,639,193, entitled “Therapeutic Agent Delivery Device with Convergent Lumen,” issued on May 5, 2020, the disclosure of which is incorporated by reference herein.

II. Example of Method for Delivery of Therapeutic Agent from Trans-Retinal Approach

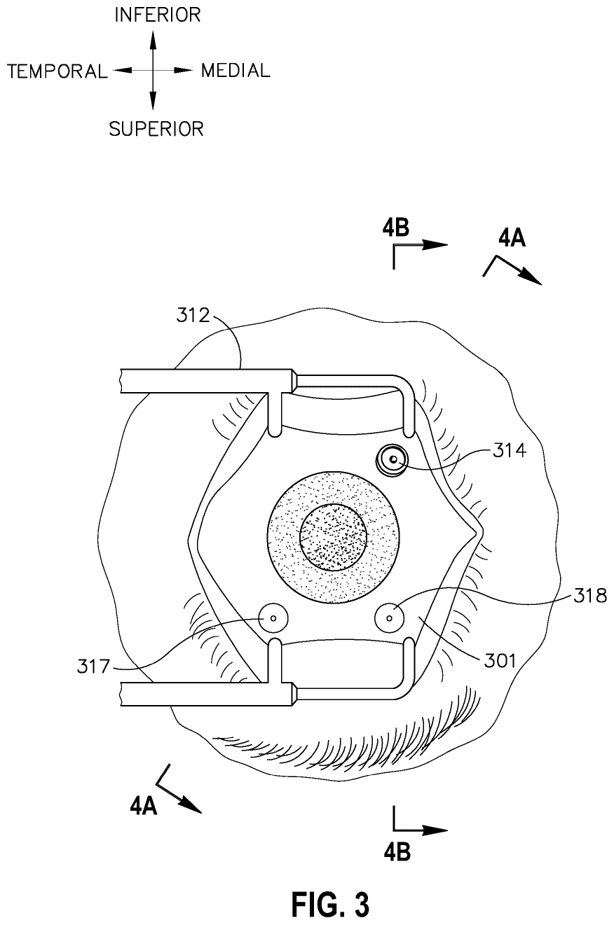

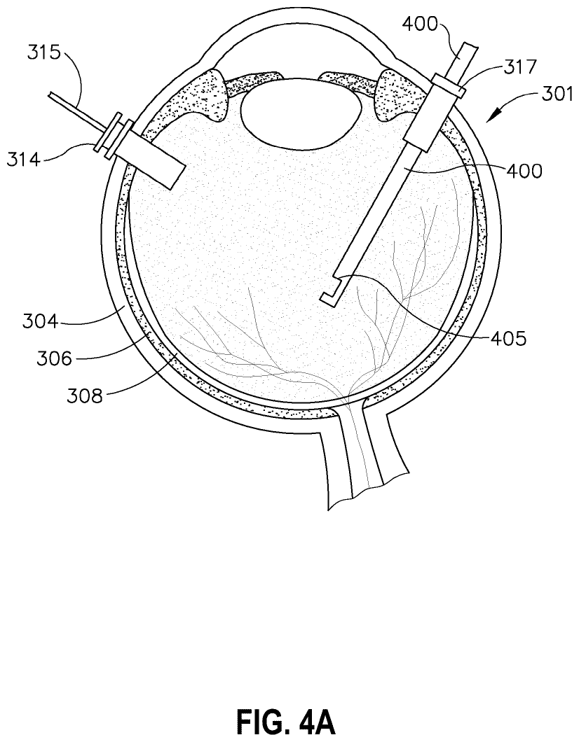

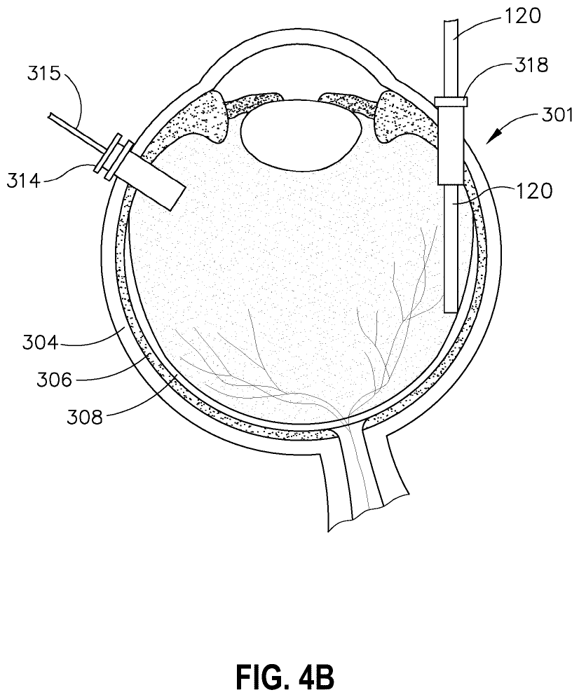

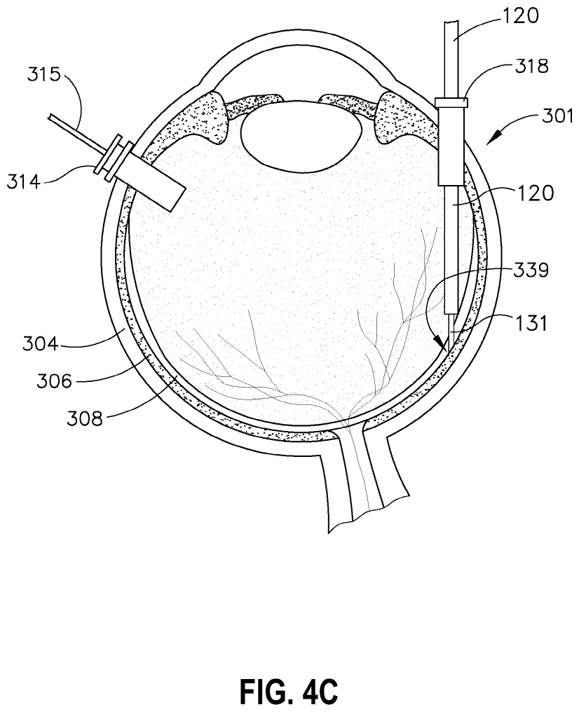

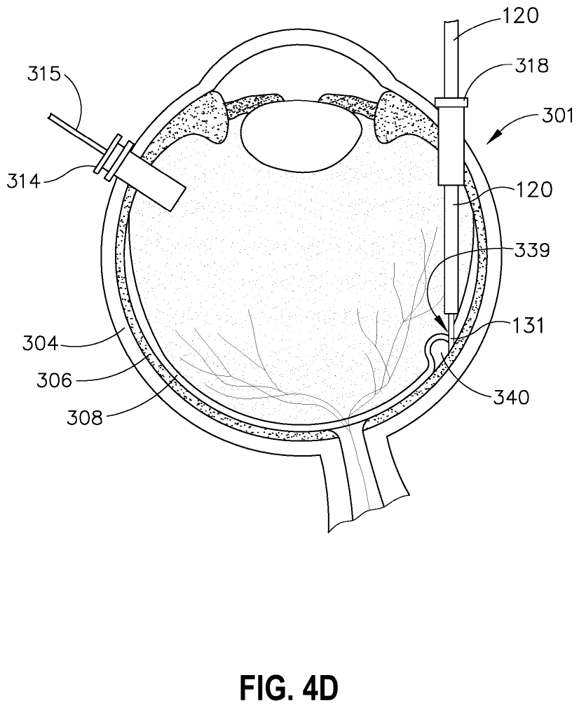

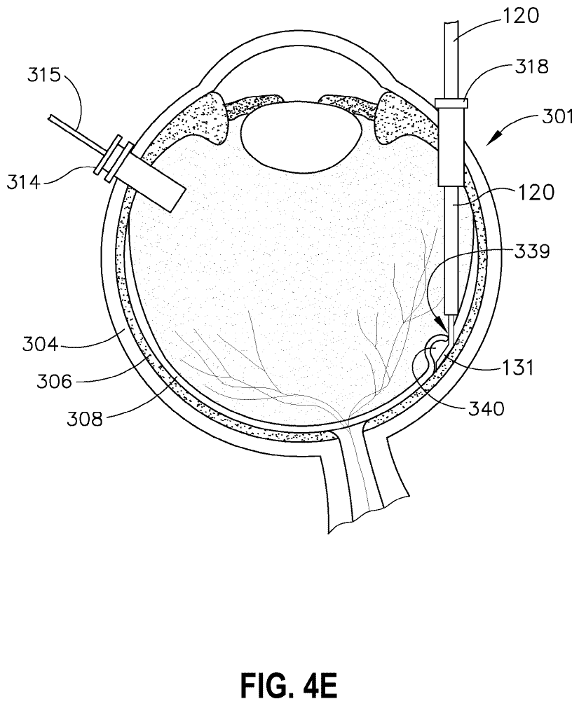

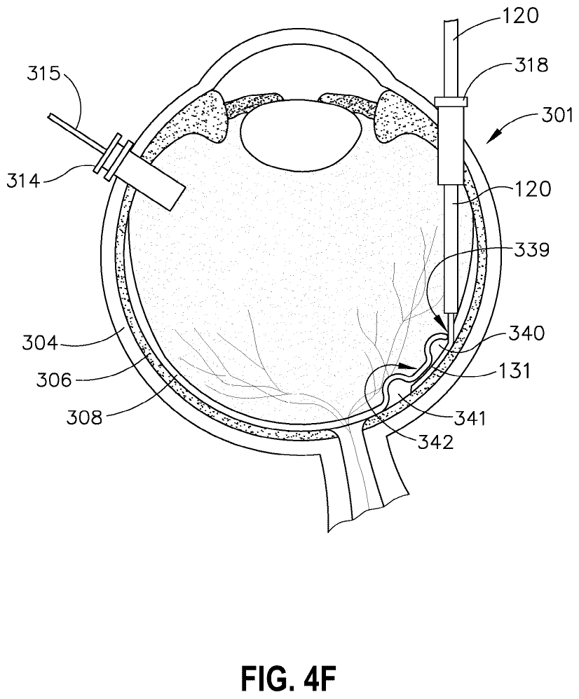

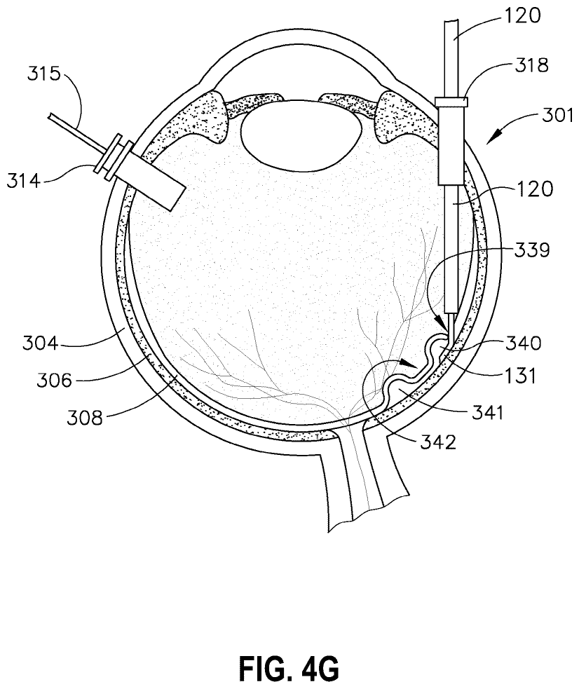

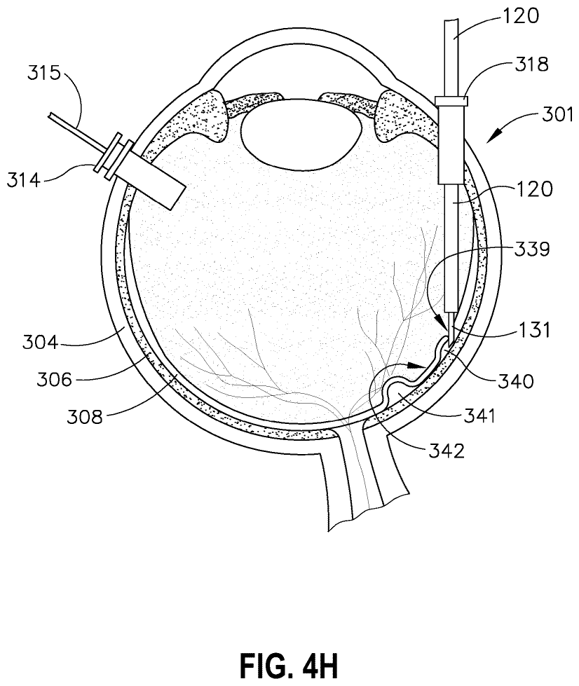

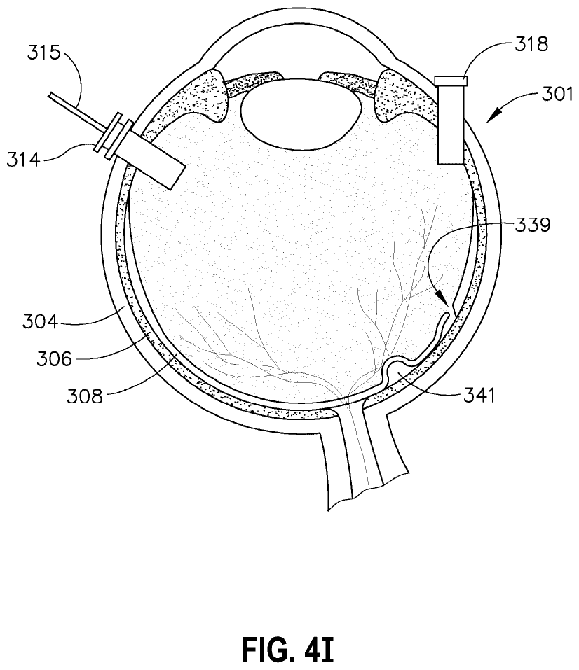

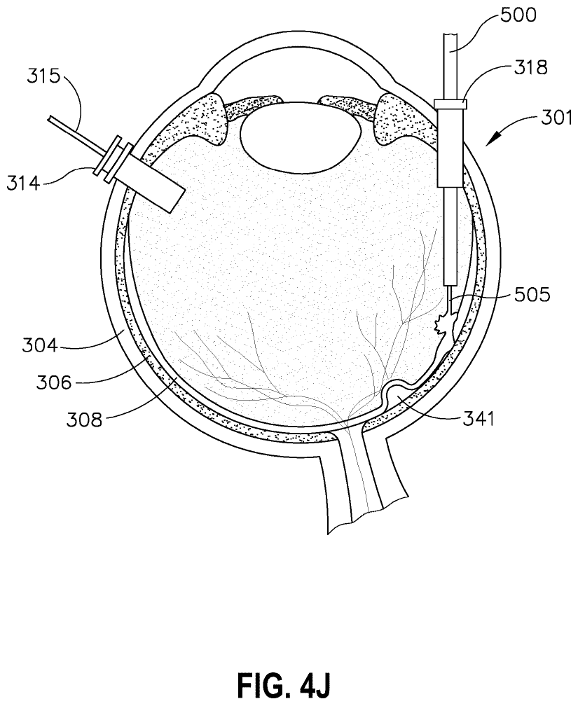

FIGS. 3-4J show a method of using instrument (110) to provide for an ipsilateral or transvitreal administration of therapeutic agent (341) to the subretinal space of a patient.

As can be seen in FIG. 3, the procedure begins by an operator immobilizing tissue surrounding a patient's eye (301) (e.g., the eyelids) using speculum (312), and/or any other instrument suitable for immobilization. While immobilization is described herein with reference to tissue surrounding eye (301), it should be understood that eye (301) itself may remain free to move. Once the tissue surrounding eye (301) has been immobilized, an eye chandelier port (314) is inserted into eye (301) to provide intraocular illumination when the interior of eye (301) is viewed through the pupil. In the present example, eye chandelier port (314) is positioned in the inferior medial quadrant. Additionally, a vitrectomy port (317) is inserted into eye (301) to provide access to perform a standard three port pars plana core vitrectomy. In the present example, vitrectomy port (317) is positioned in the superior temporal quadrant. Also, third port (318) is inserted into eye (301) to provide access for device to administer therapeutic fluid (341) to the subretinal space of eye (301). In the present example, third port (318) is positioned in the superior medial quadrant. Various suitable forms that ports (314, 317, 318) may take will be apparent to those skilled in the art in view of the teachings herein.

As can be seen in FIG. 4A, eye chandelier port (314) is positioned to direct light onto the interior of eye (314) to illuminate at least a portion of the retina (308) (e.g., including at least a portion of the macula). As will be understood, such illumination corresponds to an area of eye (301) that is being targeted for delivery of therapeutic agent (341). An optical fiber (315) has been inserted into port (314) to provide the illumination. A microscope may optionally be utilized to visually inspect the eye to confirm proper positioning of eye chandelier port (314) relative to the target site. Although FIG. 4A shows a particular positioning of eye chandelier port (314), it should be understood that eye chandelier port (314) may have any other positioning as will be apparent to those skilled in the art in view of the teachings herein. Additionally, a conventional vitrectomy instrument (400) is inserted through vitrectomy port (317) to perform a conventional three port pars plana core vitrectomy procedure. After completion of a conventional three port pars plana core vitrectomy procedure, vitrectomy instrument (400) is removed.

After or before the conventional three port pars plana core vitrectomy procedure is completed, instrument (110) may be prepared for subretinal administration of therapeutic fluid (341). By way of example only, the preparation of instrument (110) may include coupling luer fitting (195) with a source of therapeutic fluid (341) and coupling luer fitting (196) with a source leading bleb (340) fluid. Next, the fluid sources may be pressurized to prime the fluid communicating components leading up to and including the lumen of inner cannula (131) until a drop of leading bleb fluid (340) or therapeutic fluid (341) exits the distal end of inner cannula (131). In some instances, the source of therapeutic fluid (341) is pressurized first, followed by pressurization of the source of leading bleb (340) fluid. In some other instances, the source of leading bleb (340) fluid is pressurized first, followed by pressurization of the source of therapeutic fluid (341). In some other instances, both fluid sources are pressurized at substantially the same time. It should also be understood that, in some instances, an air gap may be provided between leading bleb (340) and therapeutic fluid (341) to prevent therapeutic fluid (341) from mixing with leading bleb (340). Various suitable ways in which instrument (110) may be primed with leading bleb (340) fluid, therapeutic fluid (341), and/or an intentional air gap will be apparent to those skilled in the art in view of the teachings herein.

After instrument (110) has been primed, outer cannula (120) is inserted into third port (318) as shown in FIG. 4B. When outer cannula (120) is inserted into third port (318), slider (200) is in a proximal position as shown in FIG. 2A, thereby ensuring that the distal end of inner cannula (131) is positioned within the interior of outer cannula (120). Outer cannula (120) is inserted to the point where the distal end of outer cannula (120) is positioned adjacent to the interior of retina (308).

Once the distal end of outer cannula (120) is positioned adjacent to interior of retina (308), slider (200) is slid distally such that the distal end of inner cannula (131) is exposed from the distal end of fixed outer cannula (120). Inner cannula (131) thus pierces the retina (308) to define a subretinal entry point (339) as shown in FIG. 4C.

Once the distal end of inner cannula (131) is positioned at a first location in the subretinal space via the subretinal entry point (339), the fluid source of leading bleb (430) fluid is pressurized. This pressurization drives the fluid through the lumen of inner cannula (131) to form leading bleb (340) within the subretinal space, between the retina (308) and the choroid (306), as shown in FIG. 4D.

Once leading bleb (340) is formed at the first location in the subretinal space, inner cannula (131) is advanced further distally via actuation of slider (200) so that inner cannula (131) is advanced in an inferior direction along the coronal plane and in a posterior direction along the sagittal plane as shown in FIGS. 4E-4F. In particular, inner cannula (131) first travels between leading bleb (340) and the choroid (306) (FIG. 4E) and then past leading bleb (340) into the space between the retina (308) and the choroid (306) (FIG. 4F). This advancement of inner cannula (131) thus defines a pathway (342) between the retina (308) and the choroid (306).

Once inner cannula (131) reaches a second location in the subretinal space at the end of pathway (342), therapeutic agent (341) is injected in the subretinal area by pressurizing the fluid source of therapeutic agent (341) to drive therapeutic agent (341) distally out through the lumen of inner cannula (131) as shown in FIG. 4F.

Once the proper amount of therapeutic fluid (341) has been delivered to the second location, slider (200) is retracted proximally, thereby retracting inner cannula (131) back to the first location as shown in FIG. 4G. While inner cannula (131) is being retracted from pathway (342), the leading bleb (340) fluid may again be pressurized to release leading bleb (340) fluid within pathway (342) to help seal in therapeutic fluid (341) at the second location.

Once inner cannula (131) has been retracted from pathway (342) as shown in FIG. 4G, negative pressure may be induced through either supply tube (190, 191) within inner cannula (131) to aspirate leading bleb (340) as shown in FIG. 4H. Optionally, negative pressure may be induced to aspirate leading bleb (340) through other known means in the art, such as with a separate flextip inserter (MedOne). The separate flextip inserter or other conventional instrument may be inserted after removing instrument (110) from the eye (301). In addition to or in lieu of aspirating leading bleb (340), air or other fluid may be used to effectively tamponade the bleb (340) in the subretinal space.

After leading bleb (340) has been aspirated or tamponaded, instrument (110) is removed from third port (318) as shown in FIG. 4I. Thereafter, as shown in FIG. 4J, a laser retinopexy tool (500) is then inserted into third port (318) and is used to seal the subretinal entry point (339). Alternatively, any other suitable instruments or techniques may be used to seal the subretinal entry point (339).

It should be understood from the foregoing that instrument (110) may be used to deliver two different kinds of fluids (e.g., leading bleb (340) fluid and therapeutic agent (341)) to the subretinal space without having to withdraw inner cannula (131) from the subretinal space between the act of delivering the first fluid (e.g., leading bleb (340) fluid) and the second fluid (e.g., therapeutic agent (341)).

### III. Examples of Transvitreal Subretinal Injection Systems

In some instances, it may be desirable to provide outer cannula (120) with a tapered distal tip, such as for assisting with insertion of outer cannula (120) into trocar port (318) and/or for assisting with maintaining centration of inner cannula (131) relative to outer cannula (120) during translation of inner cannula (131) relative to outer cannula (120) (e.g., by contacting or otherwise guiding inner cannula (131)). It will be appreciated that maintaining centration of inner cannula (131) relative to outer cannula (120) may allow inner cannula (131) to have a relatively high stiffness, particularly when inner cannula (131) is only slightly extended out of outer cannula (120), while still allowing inner cannula (131) to have sufficient flexibility to travel along pathway (342) as inner cannula (131) is extended further out of outer cannula (120). Such relatively high stiffness as inner cannula (131) is initially extended out of outer cannula (120) may promote controlled piercing of the retina (308) by inner cannula (131) to define subretinal entry point (339) at a desired location and/or controlled advancement of inner cannula (131) following such piercing of the retina (308), such as between leading bleb (340) and the choroid (306).

In addition, or alternatively, it may be desirable to mount the fluid sources that are coupled with luer fittings (195, 196) in a manner which restricts movement of the fluid sources relative to instrument (110). For example, it may be desirable to mount the fluid sources in a manner which prevents inadvertent movement of the fluid sources in a direction away from instrument (110) which may otherwise result in tugging of instrument (110) by the fluid sources via supply tubes (190, 191), to thereby allow for precise manipulation of instrument (110). In addition, or alternatively, it may be desirable to provide a fluid selection device that enables rapid switching between pumping of fluids (340, 341) to the corresponding supply tubes (190, 191), and/or to position the fluid selection device remotely relative to the fluid sources themselves.

Each of the examples of systems (1000, 2000) described below may function in this manner and may be used to deliver bleb fluid (340) and therapeutic agent (341) to the subretinal space from a transvitreal, trans-retinal approach.

### A. First Example of Transvitreal Subretinal Injection System

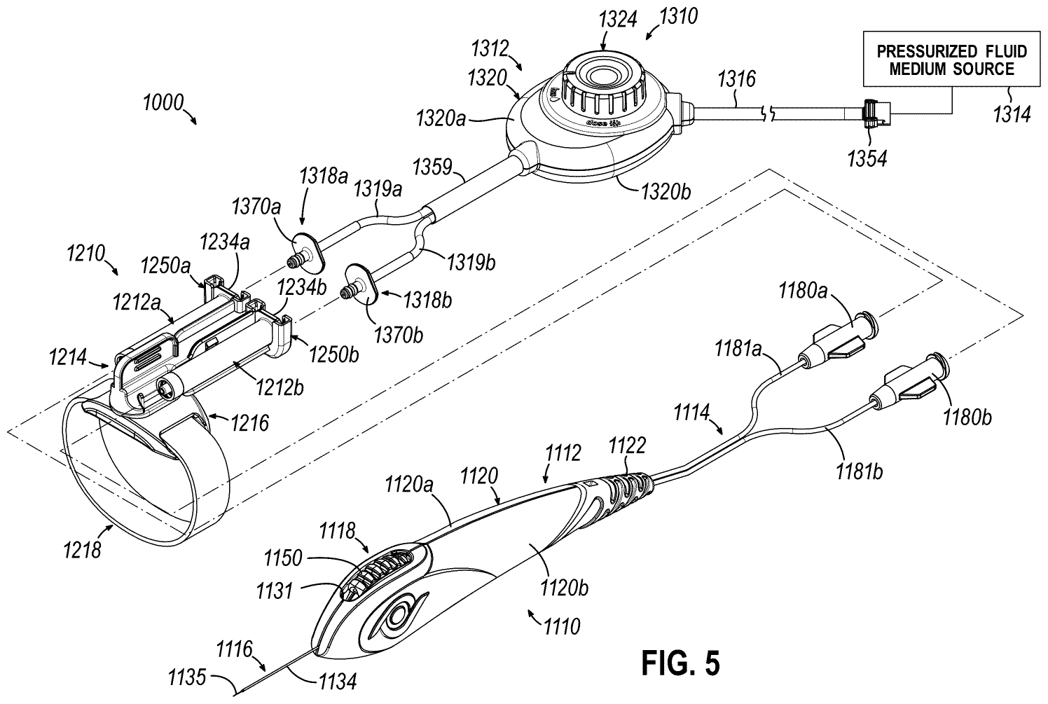

FIG. 5 shows an example of a system (1000) that comprises a fluid injection instrument (1110), a fluid source assembly (1210), and a pressure control delivery assembly (1310) for delivering one or more fluids during a procedure to treat an ocular condition, such as the subretinal delivery of therapeutic agent (341) described above.

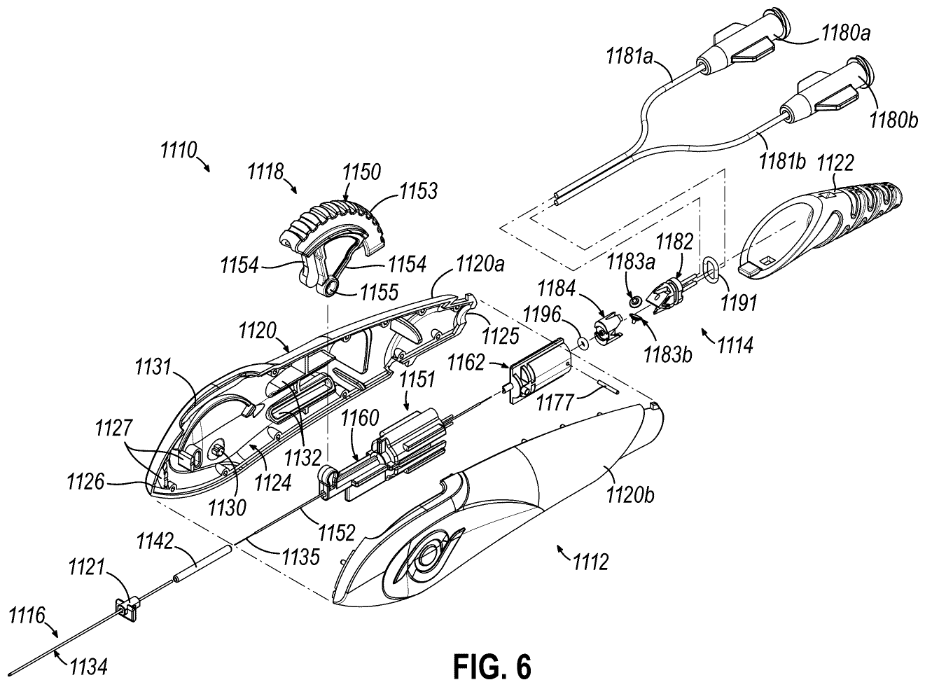

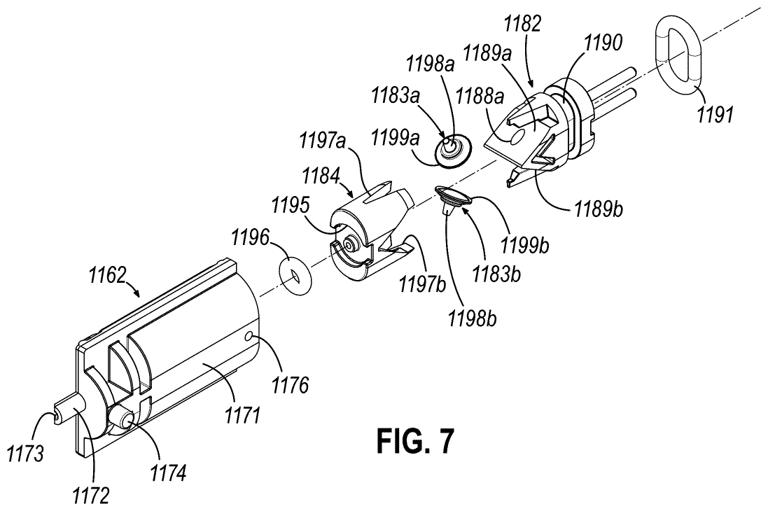

Referring primarily to FIGS. 6-12D, with continuing reference to FIG. 5, fluid injection instrument (1110) may be similar to instrument (110) described above, except as otherwise described below. In this regard, fluid injection instrument (1110) of the present example includes a handle assembly (1112), a fluid supply assembly (1114), a fluid delivery assembly (1116), and an actuation assembly (1118).

Handle assembly (1112) of this example includes a rigid handle body (1120), a cannula hub (1121), and a strain relief (1122). Handle body (1120) is sized and configured to be grasped and operated by a single hand of an operator (e.g., either a left hand or a right hand), such as via a power grip, a pencil grip, or any other suitable kind of grip. In the example shown, handle body (1120) is formed from first and second shells (e.g., halves) (1120a, 1120b) fixedly coupled to each other along a longitudinal centerline of handle body (1120) via corresponding pairs of engagement features (e.g., interlocking detents and indents), such that shells (1120a, 1120b) collectively define handle body (1120). Shells (1120a, 1120b) may each be manufactured via 3D printing, injection molding, investment casting, machining, and/or any other suitable manufacturing techniques, and subsequently assembled to each other to form handle body (1120). It will be appreciated that handle body (1120) may be formed in any other suitable manner. For example, shells (1120a, 1120b) may be integrally formed together with each other as a unitary (e.g., monolithic) piece to define handle body (1120), such that the corresponding pairs of engagement features may be omitted.

As shown, handle body (1120) defines an interior cavity (1124) that is configured to at least partially house various components of fluid supply assembly (1114), fluid delivery assembly (1116), and actuation assembly (1118). In this regard, handle body (1120) of the present example also includes a proximal bore (1125) configured to receive a portion of fluid supply assembly (1114) and a distal bore (1126) configured to receive a portion of fluid delivery assembly (1116), such that fluid supply assembly (1114) may extend out of interior cavity (1124) via proximal bore (1125) and fluid delivery assembly (1116) may extend out of interior cavity (1124) via distal bore (1126). In the example shown, a pair of internal bridges (1127) extend laterally across interior cavity (1124) and include respective central bores (1128) configured to receive a portion of fluid delivery assembly (1116) such that bridges (1127) may provide support to fluid delivery assembly (1116) within interior cavity (1124). In the example shown, cannula hub (1121) is fixedly coupled to handle body (1120) within interior cavity (1124) and includes a central bore (1129) configured to receive a portion of fluid delivery assembly (1116) such that cannula hub (1121) may provide support to fluid delivery assembly (1116) within interior cavity (1124).

Handle body (1120) of the present example also includes a pair of laterally-opposed pivot pins (1130) (one shown) extending laterally inwardly and configured to be pivotably received by corresponding portions of actuation assembly (1118), as well as an upper slot (1131) positioned generally above pivot pins (1130) and configured to expose a corresponding portion of actuation assembly (1118) to an exterior of handle body (1120), as described in greater detail below. In the example shown, handle body (1120) further includes a plurality of (e.g., four) longitudinal channels (1132) (two shown) configured to slidably receive corresponding portions of actuation assembly (1118).

As shown, strain relief (1122) is coupled to a proximal end of handle body (1120) and defines a passageway (1133) in communication with proximal bore (1125) to direct a proximal portion of fluid supply assembly (1114) proximally and/or transversely away from interior cavity (1124). In some versions, strain relief (1122) may comprise a flexible material.

Fluid delivery assembly (1116) of the present example includes an outer cannula (1134) and an inner cannula (1135). Outer cannula (1134) is similar to outer cannula (120) described above except as otherwise described below, and inner cannula (1135) is similar to inner cannula (131) described above except as otherwise described below. In this regard, outer and inner cannulas (1134, 1135) may be used together with the rest of instrument (1110) in a similar way as outer and inner cannulas (120, 131) to perform the same procedure described above in connection with FIGS. 4A-4J. Outer cannula (1134) is fixed relative to handle body (1120) and is sized and configured to pass through a trocar port (318) that is inserted into the eye (301) of a patient. Inner cannula (1135) is longitudinally slidable relative to outer cannula (1134) and handle body (1120) via actuation assembly (1118), as will be described in greater detail below. Outer cannula (1134) may be substantially rigid, while inner cannula (1135) may be flexible. In some versions, outer cannula (1134) may comprise a metallic material, such as surgical stainless steel. In addition, or alternatively, inner cannula (1135) may comprise a polymeric material, such as polyimide. Other suitable materials that may be used to form outer cannula (1134) and/or inner cannula (1135) will be readily apparent to those skilled in the art in view of the teachings herein.

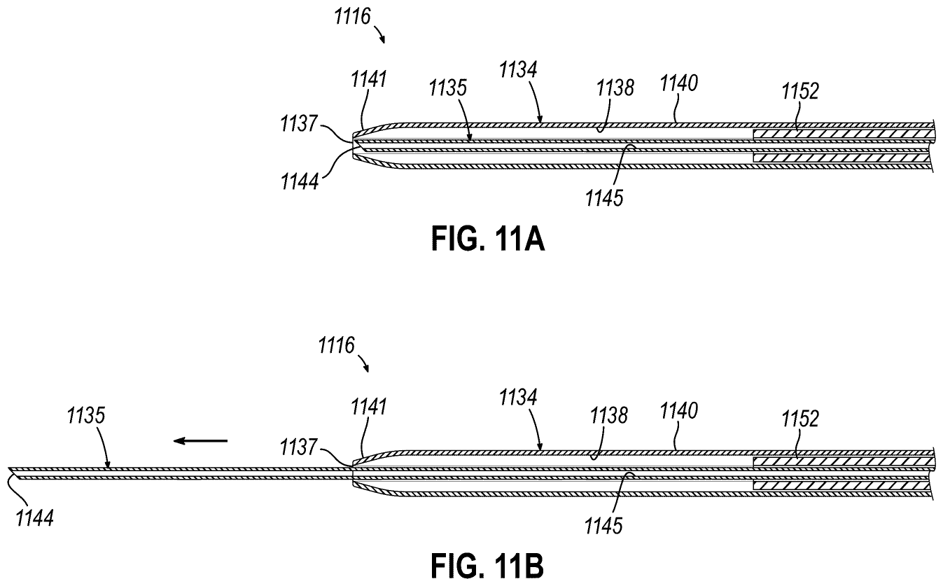

In the example shown, outer cannula (1134) extends between an open proximal end (1136) and an open distal end (1137); and defines an outer lumen (1138) extending therebetween for slidably receiving inner cannula (1135) and a portion of actuation assembly (1118). As best seen in FIGS. 11A-11B, outer cannula (1134) includes a generally cylindrical proximal portion (1140) extending distally from proximal end (1136) and a generally frustoconical distal portion (1141) tapering radially inwardly from proximal portion (1140) to distal end (1137), such that distal portion (1141) may have at least one cross-dimension (e.g., diameter) smaller than that of proximal portion (1140), and may thereby be configured to assist with guiding insertion of outer cannula (1134) into trocar port (318). For example, distal portion (1141) may be sized and configured to be funneled into trocar port (318) to thereby direct proximal portion (1140) into trocar port (318).

In addition, or alternatively, the tapered configuration of distal portion (1141) may cause outer lumen (1138) to be constricted at distal portion (1141), such that distal end (1137) defines a smaller opening than that defined by proximal end (1136). The relatively small opening defined by distal end (1137) may assist with maintaining centration of inner cannula (1135) relative to outer cannula (1134) (e.g., ensuring a coaxial relationship between inner cannula (1135) and outer cannula (1134) during translation of inner cannula (1135) relative to outer cannula (1134), such as by contacting or otherwise guiding inner cannula (1135) out through distal end (1137). Such centration of inner cannula (1135) relative to outer cannula (1134) may allow inner cannula (1135) to have a relatively high stiffness, at least when inner cannula (1135) is only slightly extended out of outer cannula (1134), such as during piercing of the retina (308), for example.

While distal portion (1141) of the present example tapers radially inwardly from proximal portion (1140) to distal end (1137), distal portion (1141) may alternatively curve radially inwardly from proximal portion (1140) to distal end (1137). While proximal and distal portions (1140, 1141) of the present example are integrally formed together as a unitary (e.g., monolithic) piece to define outer cannula (1134), proximal and distal portions (1140, 1141) may alternatively be separately formed from each other as distinct pieces and coupled to each other to define outer cannula (1134).

Any one or more of distal bore (1126) and/or central bores (1128, 1129) of handle assembly (1112) may be configured to receive proximal portion (1140) of outer cannula (1134) for fixedly securing outer cannula (1134) to handle body (1120). For example, one or more of distal bore (1126) and/or central bores (1128, 1129) may be configured to frictionally engage a generally cylindrical outer surface of proximal portion (1140) of outer cannula (1134) for fixedly securing outer cannula (1134) to handle body (1120). As shown, a fixed cannula support tube (1142) is secured to a proximal portion (1140) of outer cannula (1134) and is disposed at least partially within a portion of actuation assembly (1118) for further supporting outer cannula (1134).

In the example shown, inner cannula (1135) extends between an open proximal end (1143) and an open distal end (1144); and defines an inner lumen (1145) extending therebetween for directing fluid therethrough. As best seen in FIGS. 11A-11B, distal end (1144) of inner cannula (1135) is beveled such that distal end (1144) is oriented at an angle relative to a longitudinal axis of inner cannula (1135). The angle at which distal end (1144) is oriented relative to the longitudinal axis of inner cannula (1135) may be about 45 degrees, for example. In this manner, distal end (1144) of inner cannula (1135) may be configured to be oriented generally parallel to the retinal pigment epithelium (RPE) of the eye (301) when inner lumen (1145) is used to pierce the retina (308) to define the subretinal entry point (339) and/or to form leading bleb (340) within the subretinal space, which may reduce the risk of distal end (1144) piercing or otherwise imparting undesired trauma to the RPE and/or improve the formation of leading bleb (340).

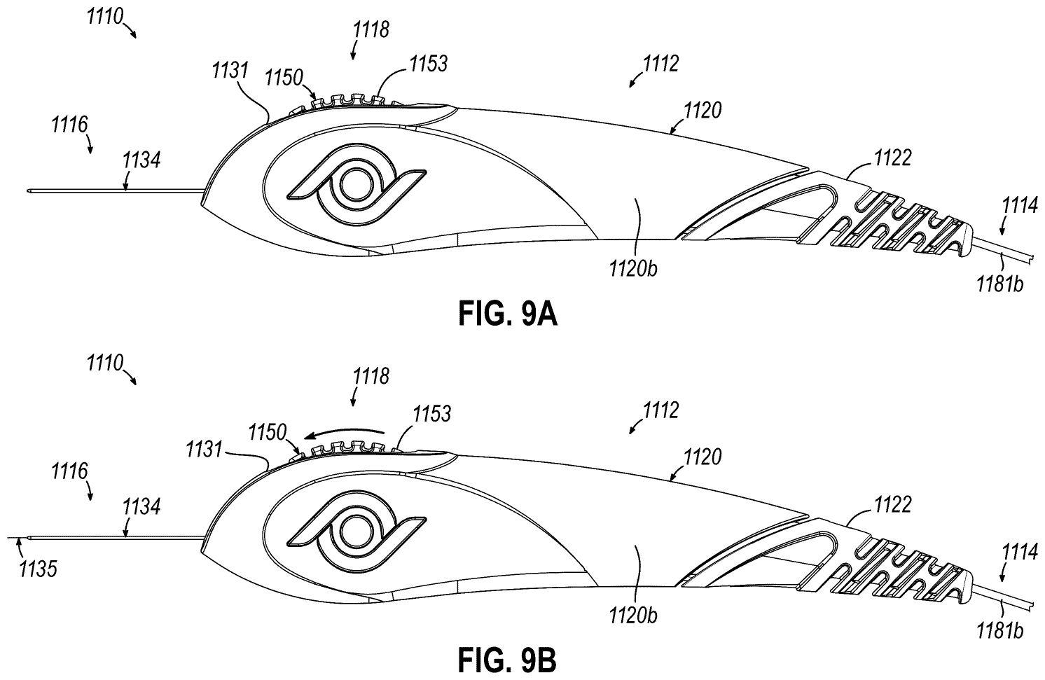

Actuation assembly (1118) of the present example includes a pivotable scroll wheel (1150), a translatable cannula sled (1151), and a translatable cannula support tube (1152) configured to cooperate with each other to drive translation of inner cannula (1135) relative to outer cannula (1134). In the example shown, scroll wheel (1150) is pivotably coupled to handle body (1120) and includes a generally arch-shaped grip portion (1153) and a pair of laterally-opposed arms (1154) extending generally downwardly from grip portion (1153). Scroll wheel (1150) of the present example also includes a pair of pivot bores (1155) (one shown), each facing laterally outwardly from a lower portion of a respective arm (1154) and configured to pivotably receive a corresponding pivot pin (1130) of handle body (1120) for pivotably coupling scroll wheel (1150) to handle body (1120), such that scroll wheel (1150) may be pivotable about a pivot axis collectively defined by pivot pins (1130) and pivot bores (1155) between an unactuated state (FIGS. 9A, 10A, and 12A) and at least one actuated state, such as a fully actuated state (FIGS. 9B, 10B, and 12B). In this regard, grip portion (1153) may be at least partially exposed to an exterior of handle body (1120) via upper slot (1131) to enable the operator to access and manipulate grip portion (1153) between the unactuated and fully actuated states with the operator's thumb, for example. In some versions, handle body (1120) may include one or more stop elements configured to selectively engage corresponding portions of scroll wheel (1150) when scroll wheel (1150) is pivoted to the unactuated and/or fully actuated states to prevent further pivoting of scroll wheel (1150) beyond the unactuated and/or fully actuated states.

In the example shown, scroll wheel (1150) also includes a pair of parallel flanges (1156) extending generally downwardly from grip portion (1153) between arms (1154) and having confronting, generally planar cam surfaces (1157) that are spaced apart from each other to define a socket (1158) that is configured to receive a portion of cannula sled (1151).

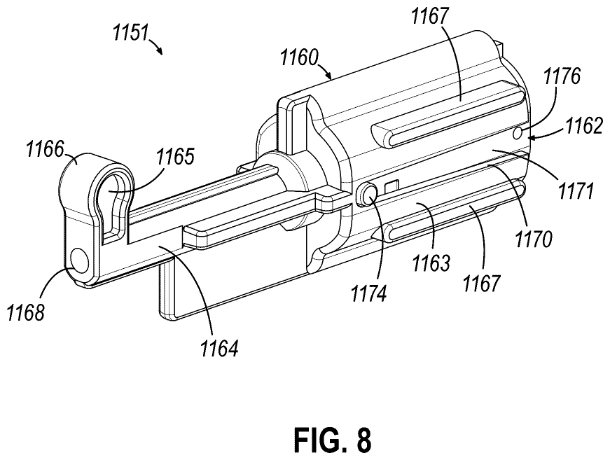

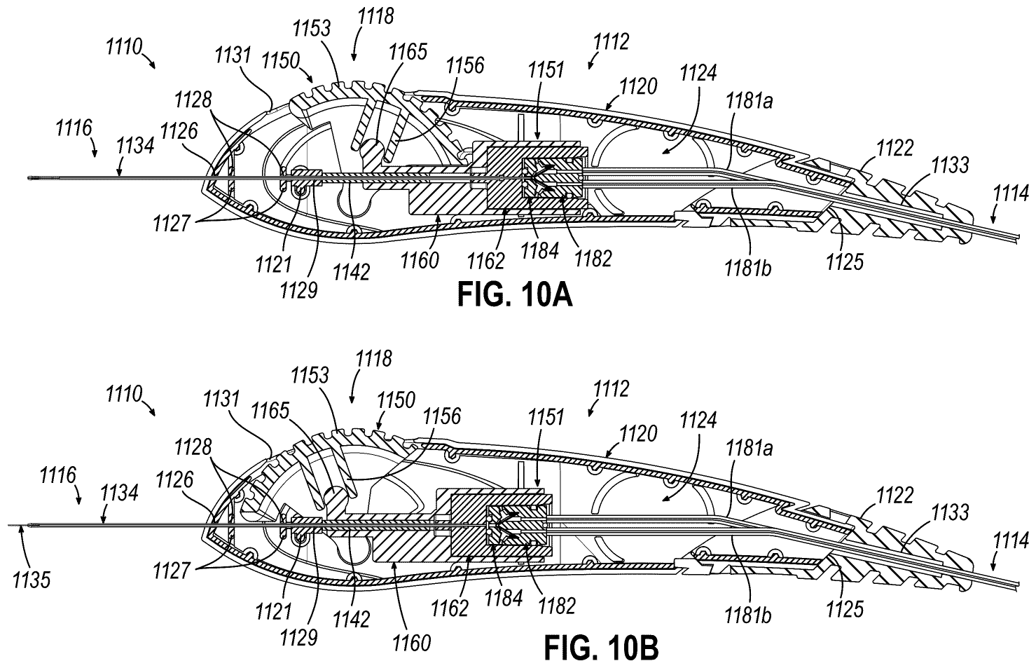

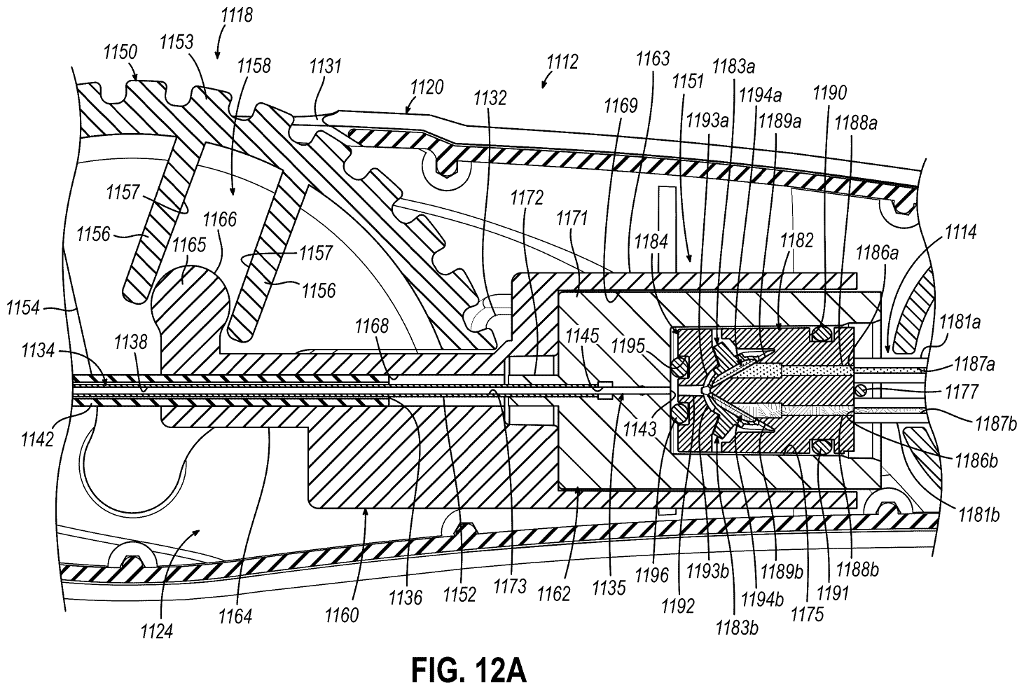

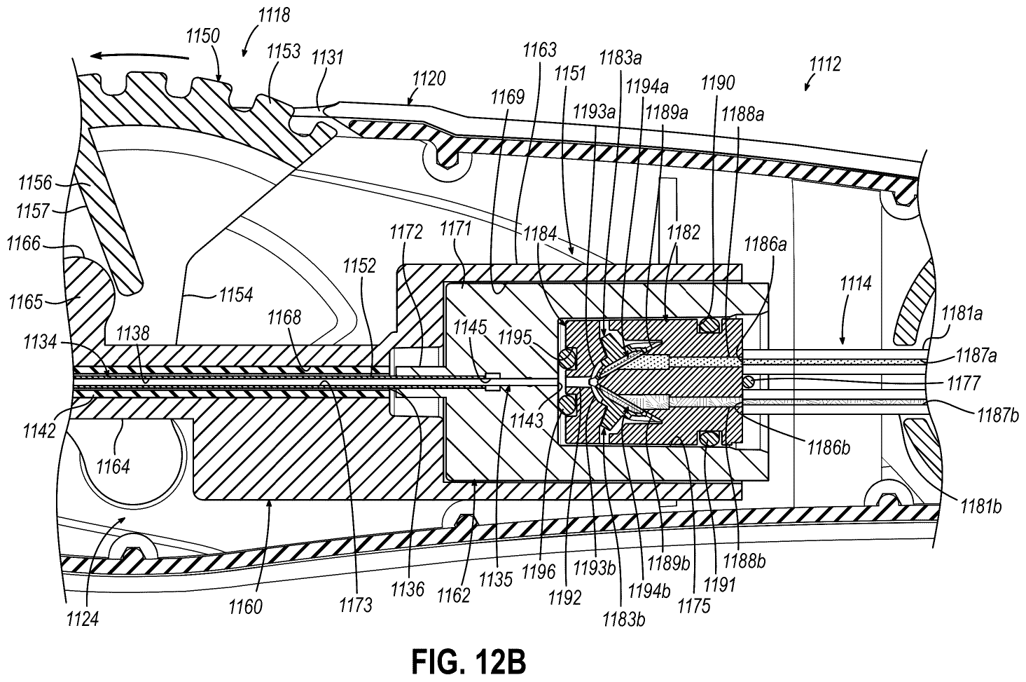

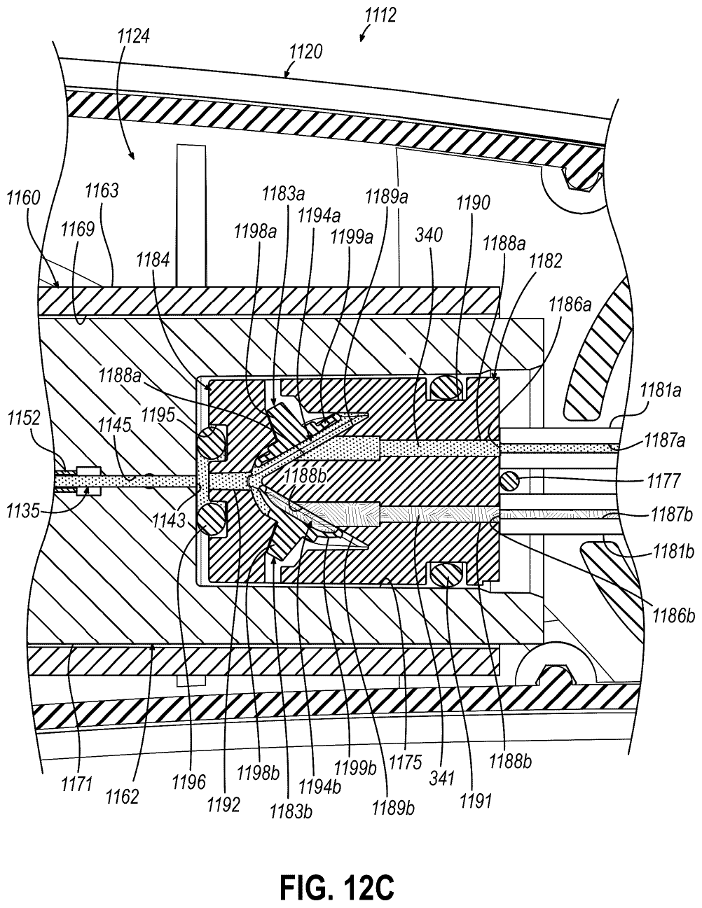

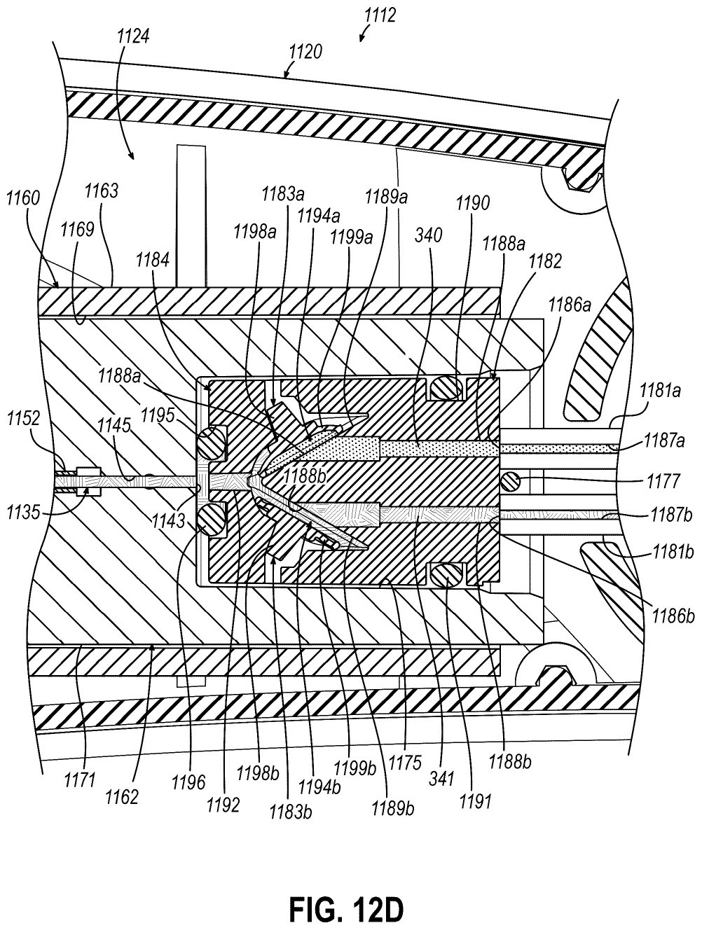

In this regard, cannula sled (1151) of the present example is translatably coupled to handle body (1120) and includes a sled housing (1160) and a connection member (1162). As best shown in FIG. 8, sled housing (1160) includes a drum (1163) and a yoke (1164) extending distally from drum (1163) and having a generally bulbous distal hitch (1165) extending generally upwardly for receipt within socket (1158) of scroll wheel (1150). In the example shown, hitch (1165) has a generally C-shaped cam surface (1166) configured to be cammingly engaged by one or both planar cam surfaces (1157) of scroll wheel (1150) to convert pivoting of scroll wheel (1150) into translation of cannula sled (1151), such that cannula sled (1151) may be longitudinally translatable between a proximal position (FIGS. 10A and 12A) and a distal position (FIGS. 10B and 12B) in response to pivoting of scroll wheel (1150) between the unactuated and fully actuated states. Drum (1163) of the present example includes a plurality of (e.g., four) longitudinal rails (1167) (two shown) configured to be slidably received within corresponding longitudinal channels (1132) of handle body (1120), such that the interaction between channels (1132) and rails (1167) may guide longitudinal translation of cannula sled (1151) between the proximal and distal positions during pivoting of scroll wheel (1150) between the unactuated and fully actuated states.

As shown, yoke (1164) of sled housing (1160) also includes a central bore (1168) configured to slidably receive cannula support tube (1142) such that outer cannula (1134) of fluid delivery assembly (1116) may be supported by cannula sled (1151) via cannula support tube (1142) without interfering with longitudinal translation of cannula sled (1151). In this regard, central bore (1168) may have an inner cross-dimension (e.g., diameter) at least slightly greater than an outer cross-dimension (e.g., diameter) of cannula support tube (1142). In the example shown, proximal end (1136) of outer cannula (1134) is disposed within central bore (1168). Drum (1163) of sled housing (1160) also includes a chamber (1169) disposed proximal of central bore (1168) and configured to securely retain connection member (1162). In this regard, a longitudinal alignment slot (1170) extends distally from a proximal end of drum (1163) through a sidewall of drum (1163) for assisting with promoting proper alignment of connection member (1162) relative to sled housing (1160) when connection member (1162) is loaded into chamber (1169) of drum (1163).

Connection member (1162) of the present example includes a drum (1171) and a tube grip (1172) extending distally from drum (1171). As shown, tube grip (1172) of connection member (1162) includes a central generally C-shaped recess (1173) configured to securely receive cannula support tube (1152) to fix cannula support tube (1152) against movement relative to connection member (1162), such that cannula support tube (1152) may be longitudinally translatable together with cannula sled (1151). In this regard, central recess (1173) has an inner cross-dimension (e.g., diameter) substantially equal to an outer cross-dimension (e.g., diameter) of cannula support tube (1152) to provide a friction fit therebetween. In some versions, a lateral cover (not shown) may be secured to a side of connection member (1162) to capture cannula support tube (1152) within central recess (1173). For example, such a cover may include a central generally C-shaped recess which may align with central recess (1173) to collectively define a central bore configured to securely receive cannula support tube (1152).

In the example shown, cannula support tube (1152) is slidably received within outer lumen (1138) of outer cannula (1134); and is configured to securely receive inner cannula (1135) to fix inner cannula (1135) against movement relative to cannula support tube (1152) (and thus relative to connection member (1162)), such that inner cannula (1135) may be longitudinally translatable together with cannula support tube (1152) and cannula sled (1151). In the example shown, proximal end (1136) of outer cannula (1134) is spaced apart from tube grip (1172) by a sufficient distance to permit translation of cannula sled (1151) between the proximal and distal positions without risk of collision between tube grip (1172) and proximal end (1136) of outer cannula (1134). Drum (1171) of connection member (1162) includes a radially-outwardly extending alignment protrusion (1174) configured to be slidably received within alignment slot (1170) of sled housing (1160) for maintaining proper alignment of connection member (1162) relative to sled housing (1160). Drum (1171) also includes a chamber (1175) disposed proximal of central recess (1173) and configured to securely retain at least a portion of fluid supply assembly (1114). In this regard, drum (1171) includes a pair of laterally-opposed bores (1176) (one shown) configured to selectively receive a retention pin (1177) for retaining various components of fluid supply assembly (1114) within chamber (1175).

Fluid supply assembly (1114) of the present example includes first and second luer fittings (1180a, 1180b), first and second fluid supply tubes (1181a, 1181b), a fluid supply junction (1182), first and second self-actuating check valves (1183a, 1183b), and a valve retainer (1184). In the example shown, each fluid supply tube (1181a, 1181b) extends between an open proximal end (not shown) and an open distal end (1186a, 1186b), and defines a lumen (1187a, 1187b) extending therebetween for directing fluid therethrough. The open proximal end of each fluid supply tube (1181a, 1181b) is fluidly connected to the respective luer fitting (1180a, 1180b), and each open distal end (1186a, 1186b) is fluidly connected to fluid supply junction (1182), as described in greater detail below. In the example shown, fluid supply tubes (1181a, 1181b) both extend through proximal bore (1125) of handle body (1120) and passageway (1133) of strain relief (1122), such that open distal ends (1186a, 1186b) are positioned within interior cavity (1124) of handle body (1120) for coupling to fluid supply junction (1182) while the open proximal ends of fluid supply tubes (1181a, 1181b) are positioned external to handle body (1120) for coupling to the respective luer fittings (1180a, 1180b). Fluid supply tubes (1181a, 1181b) may each be configured to slide through proximal bore (1125) of handle body (1120) and passageway (1133) of strain relief (1122) to accommodate translation of cannula sled (1151) relative to handle body (1120). For example, distal translation of cannula sled (1151) relative to handle body (1120) may pull open distal ends (1186a, 1186b) of fluid supply tubes (1181a, 1181b) slightly distally as shown in FIGS. 10A-10B. In some versions, supply tubes (1181a, 1181b) may be provided with sufficient slack within interior cavity (1124) of handle body (1120) such that supply tubes (1181a, 1181b) need not necessarily slide through proximal bore (1125) during translation of cannula sled (1151) relative to handle body (1120).

Luer fittings (1180a, 1180b) may connect to two different sources of fluid, such as a source of leading bleb fluid (340) and a source of therapeutic fluid (341), which may be selectively pressurized to selectively pump fluids (340, 341) to corresponding supply tubes (1181a, 1181b), as described in greater detail below. While conventional luer fittings (1180a, 1180b) are used in the present example, any other suitable kind of fittings may be used.

As shown, fluid supply junction (1182) and valve retainer (1184) are each securely retained within chamber (1175) of connection member (1162) of actuation assembly (1118). More particularly, retention pin (1177) (FIG. 6) captures fluid supply junction (1182) and valve retainer (1184) within chamber (1175). Fluid supply junction (1182) of the present example includes first and second proximal fluid supply passageways (1188a, 1188b) fluidly isolated from each other and configured to fluidly couple to open distal ends (1186a, 1186b) of first and second fluid supply tubes (1181a, 1181b), respectively. In the example shown, fluid supply junction (1182) includes first and second generally annular valve seats (1189a, 1189b) defined about open distal ends of the respective fluid supply passageways (1188a, 1188b) and angled relative to each other to define a “V” shape, the purposes of which are described below. As shown, a generally obround groove (1190) extends radially inwardly from a radially outer surface of fluid supply junction (1182), and a proximal gasket in the form of a generally obround O-ring (1191) is disposed within groove (1190) for providing a fluid-tight seal between fluid supply junction (1182) and a side of chamber (1175).

Valve retainer (1184) of the present example includes a single distal fluid supply passageway (1192) fluidly coupled to inner lumen (1145) of inner cannula (1135) and configured to selectively fluidly couple to first and second proximal fluid supply passageways (1188a, 1188b) of fluid supply junction (1182). In this regard, valve retainer (1184) also includes first and second generally dome-shaped recesses (1193a, 1193b) configured to confront first and second valve seats (1189a, 1189b), respectively, to define first and second fluid gateways (1194a, 1194b) therebetween. As shown, a generally circular groove (1195) extends proximally from a distal surface of valve retainer (1184), and a distal gasket in the form of a generally circular O-ring (1196) is disposed within groove (1195) for providing a fluid-tight seal between valve retainer (1184) and a distal end of chamber (1175).

As shown, each fluid gateway (1194a, 1194b) is configured to selectively fluidly couple a corresponding proximal fluid supply passageway (1188a, 1188b) to distal fluid supply passageway (1192) for selectively directing fluid from the corresponding proximal fluid supply passageway (1188a, 1188b) to distal fluid supply passageway (1192) through the respective fluid gateway (1194a, 1194b). In this regard, a corresponding portion of each valve (1183a, 1183b) is at least partially disposed within each fluid gateway (1194a, 1194b) for selectively placing the corresponding proximal fluid supply passageway (1188a, 1188b) into and out of fluid communication with distal fluid supply passageway (1192). To that end, valve retainer (1184) includes first and second valve retention slots (1197a, 1197b). Each valve (1183a, 1183b) includes a generally frustoconical stem (1198a, 1198b) securely received within a respective valve retention slot (1197a, 1197b), and a generally disc-shaped flexible membrane (1199a, 1199b). In some versions, fluid supply junction (1182) may capture stems (1198a, 1198b) within the respective valve retention slots (1197a, 1197b). Each disc-shaped membrane (1199a, 1199b) is bendably coupled to the corresponding stem (1198a, 1198b) (e.g., via a corresponding living hinge), such that each membrane (1199a, 1199b) is bendable between respective open and closed states.

More particularly, membrane (1199a) of first valve (1183a) is bendable between a closed state (FIGS. 12A, 12B, and 12D) and an open state (FIG. 12C). In the closed state, membrane (1199a) sealingly engages first valve seat (1189a) of fluid supply junction (1182) to prevent fluid from flowing from first proximal fluid supply passageway (1188a) through first fluid gateway (1194a) to distal fluid supply passageway (1192). In the open state, membrane (1199a) disengages first valve seat (1189a) of fluid supply junction (1182) to permit fluid to flow from first proximal fluid supply passageway (1188a) through first fluid gateway (1194a) to distal fluid supply passageway (1192). Likewise, membrane (1199b) of second valve (1183b) is bendable between a closed state (FIGS. 12A-12C) and an open state (FIG. 12D). In the closed state, membrane (1199b) sealingly engages second valve seat (1189b) of fluid supply junction (1182) to prevent fluid from flowing from second proximal fluid supply passageway (1188b) through second fluid gateway (1194b) to distal fluid supply passageway (1192). In the open state, membrane (1199b) disengages second valve seat (1189b) of fluid supply junction (1182) to permit fluid to flow from second proximal fluid supply passageway (1188b) through second fluid gateway (1194b) to distal fluid supply passageway (1192).

Each membrane (1199a, 1199b) of the present example is resiliently biased to assume the respective closed state and is configured to bend toward the respective open state in response to application of a threshold downstream-directed force to the respective membrane (1199a, 1199b), which may result from the presence of a threshold fluid pressure within the corresponding proximal fluid supply passageway (1188a, 1188b). For example, each membrane (1199a, 1199b) may be configured to immediately bend toward the respective open state in response to the fluid pressure within the corresponding proximal fluid supply passageway (1188a, 1188b) reaching the threshold fluid pressure to permit flow of fluid in the downstream direction, and may be configured to immediately resiliently return to the respective closed state in response to the fluid pressure within the corresponding proximal fluid supply passageway (1188a, 1188b) dropping below the threshold fluid pressure to prevent backflow of the fluid in the upstream direction. In this regard, each valve (1183a, 1183b) may comprise an elastomeric material, such as silicone. In some versions, each valve (1183a, 1183b) is configured as a Belleville valve, such as of the type sold by Minivalve International of Oldenzaal, the Netherlands or from any other suitable source. In addition, or alternatively, each valve (1183a, 1183b) may be configured and operable in accordance with one or more teachings of EP 1953432, entitled “Fluid Control Valve,” granted on Apr. 12, 2017.

As shown in FIG. 12C, when membrane (1199a) of first valve (1183a) is in the respective open state and membrane (1199b) of second valve (1183b) is in the respective closed state, bleb fluid (340) may flow from first luer fitting (1180a) through first lumen (1187a) of first fluid supply tube (1181a), through first proximal fluid supply passageway (1188a) of fluid supply junction (1182), through first fluid gateway (1194a), through distal fluid supply passageway (1192) of valve retainer (1184), and into inner lumen (1145) of inner cannula (1135) for delivery at beveled distal end (1144). Due to the closure of membrane (1199b) of second valve (1183b), therapeutic fluid (341) may remain substantially isolated from bleb fluid (340) within instrument (1110) during such delivery of bleb fluid (340).

As shown in FIG. 12D, when membrane (1199b) of second valve (1183b) is in the respective open state and membrane (1199a) of first valve (1183a) is in the respective closed state, therapeutic fluid (341) may flow from second luer fitting (1180b) through second lumen (1187b) of second fluid supply tube (1181b), through second proximal fluid supply passageway (1188b) of fluid supply junction (1182), through second fluid gateway (1194b), through distal fluid supply passageway (1192) of valve retainer (1184), and into inner lumen (1145) of inner cannula (1135) for delivery at beveled distal end (1144). Due to the closure of membrane (1199a) of first valve (1183a), bleb fluid (340) may remain substantially isolated from therapeutic fluid (341) within instrument (1110) during such delivery of therapeutic fluid (341).

It will be appreciated that proximal O-ring (1191) may assist with preventing fluid that is flowing through either fluid supply gateway (1194a, 1194b) from leaking proximally out of chamber (1175) of connection member (1162), and that distal O-ring (1196) may assist with preventing fluid that is flowing between distal fluid supply passageway (1192) and inner lumen (1145) of inner cannula (1135) from leaking toward the side of chamber (1175) of connection member (1162).

While Belleville valves (1183a, 1183b) are used in the present example, any other suitable kind of valves may be used, such as umbrella valves. Other suitable ways in which valves may be incorporated into instrument (1100) and/or system (1000) will be apparent to those skilled in the art in view of the teachings herein. For instance, instrument (1100) may include an integral valve assembly that is configured and operable in accordance with at least some of the teachings of U.S. Pat. No. 10,226,379, entitled “Method and Apparatus for Subretinal Administration of Therapeutic Agent,” issued on Mar. 12, 2019, the disclosure of which is incorporated by reference herein.

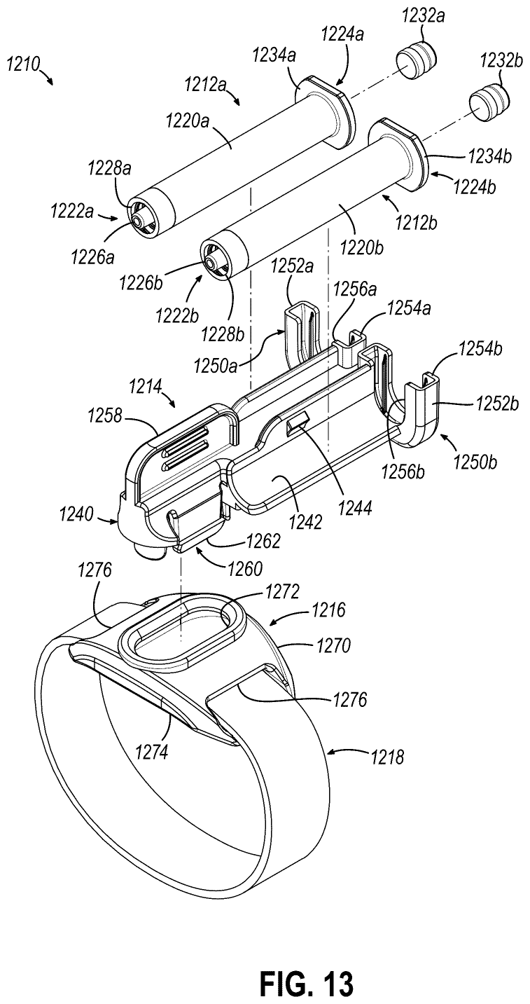

Referring now to FIG. 13, fluid source assembly (1210) of the present example includes first and second fluid sources in the form of syringes (1212a, 1212b), a syringe cradle (1214), a cradle dock (1216), and a wearable component in the form of a sterile wristband (1218). As shown, each syringe (1212a, 1212b) includes a barrel (1220a, 1220b) having a distal end (1222a, 1222b), a proximal end (1224a, 1224b), and a lumen (not shown) extending therebetween. Each distal end (1222a, 1222b) includes a first, dispensing opening (1226a, 1226b) and a threaded portion (1228a, 1228b) that enables coupling of the respective syringe (1212a, 1212b) to a needle, tubing, etc. In some versions, each threaded portion (1228a, 1228b) comprises a conventional luer fitting. In the example shown, threaded portions (1228a, 1228b) are configured to threadably engage first and second luer fittings (1180a, 1180b), respectively, for fluidly coupling the respective lumens of syringes (1212a, 1212b) with the corresponding lumens (1187a, 1187b) of supply tubes (1181a, 1181b). In this regard, the lumen of first syringe (1220a) contains bleb fluid (340) while the lumen of second syringe (1220b) contains therapeutic fluid (341). Each proximal end (1224a, 1224b) includes a second opening (not shown) that is configured to receive a corresponding plunger piston (1232a, 1232b). In some versions, the lumen of each syringe (1212a, 1212b) includes a first portion that is configured to receive the corresponding piston (1232a, 1232b), and a second, decreased cross-sectional dimension portion at the respective proximal end (1224a, 1224b). Each piston (1232a, 1232b) may be selectively advanced and retracted by fluidly coupling the lumen of the respective syringe (1212a, 1212b) with a source of pressurized air, as discussed in further detail below.

Proximal end (1224a, 1224b) of each syringe barrel (1220a, 1220b) comprises a flange (1234a, 1234b) that extends radially outwardly relative to a longitudinal axis of the respective syringe (1212a, 1212b) and that may act as a finger grip when a user holds the respective syringe (1212a, 1212b), for example. By way of example only, each syringe (1212a, 1212b) may be configured and operable in accordance with at least some of the teachings of U.S. Pat. No. 10,258,502, entitled “Therapeutic Agent Delivery Device,” issued on Apr. 16, 2019, the disclosure of which is incorporated by reference herein.

Syringe cradle (1214) of the present example includes a cradle body (1240) having a pair of parallel, longitudinal channels (1242) (one shown) configured to selectively retain respective syringe barrels (1220a, 1220b) for selectively securing syringes (1212a, 1212b) to syringe cradle (1214). In this regard, a pair of detents (1244) (one shown) each extend laterally outwardly partially over the respective channel (1242) to assist with selectively retaining the respective syringe barrel (1220a, 1220b) within the respective channel (1242), such as by providing a snap-fit engagement with the respective syringe barrel (1220a, 1220b).

In the example shown, a pair of collars (1250a, 1250b) are positioned at a proximal end of cradle body (1240) in alignment with respective channels (1242). Collars (1250a, 1250b) enable the respective syringes (1212a, 1212b) to be coupled with a system with one or more powered components as described in greater detail below. Each collar (1250a, 1250b) may be used to secure corresponding portions of pressure control delivery assembly (1310) (e.g., syringe adapters (1318a, 1318b) described below) to proximal ends (1224a, 1224b) of syringes (1212a, 1212b) and thereby prevent movement of such portions of pressure control delivery assembly (1310) relative to the respective syringes (1212a, 1212b). Each collar (1250a, 1250b) may be configured to keep such portions of pressure control delivery assembly (1310) secured to the respective syringes (1212a, 1212b) even when a pressurized medium is being communicated to either syringe (1212a, 1212b) at a fluid pressure between approximately 20 psi and approximately 40 psi. Each collar (1250a, 1250b) of the present example includes a generally U-shaped body defined by a U-shaped wall (1252a, 1252b), a first flange (1254a, 1254b) extending perpendicularly from the wall (1252a, 1252b) and an opposing second flange (1256a, 1256b) extending perpendicularly from the wall (1252a, 1252b) to define a U-shaped cavity that is sized and configured to receive at least a portion of the flange (1234a, 1234b) of the respective syringe (1212a, 1212b) and/or a corresponding portion of pressure control delivery assembly (1310) (e.g., a corresponding flange (2370a, 2370b) described below). By way of example only, each collar (1250a, 1250b) may be configured and operable in accordance with at least some of the teachings of U.S. Pat. No. 10,258,502, entitled “Therapeutic Agent Delivery Device,” issued on Apr. 16, 2019, the disclosure of which is incorporated by reference herein.

As shown, a grip tab (1258) extends upwardly from a distal end of cradle body (1240) for assisting the operator with manipulating cradle (1214), such as with selectively coupling cradle (1214) to cradle dock (1216) and/or selectively removing cradle (1214) from cradle dock (1216). A coupling member in the form of a plug (1260) extends downwardly from the distal end of cradle body (1240) for facilitating such selective coupling and removal of cradle (1214) to and from cradle dock (1216). In the example shown, plug (1260) includes a pair of laterally-opposed deflectable beams (1262) (one shown) configured to provide a snap-fit engagement with a corresponding portion of cradle dock (1216).

In this regard, cradle dock (1216) of the present example includes a generally arch-shaped plate (1270) and a central socket (1272) extending through arch-shaped plate (1270) that is configured to selectively receive plug (1260) and thereby selectively couple cradle (1214) to cradle dock (1216). For example, a periphery of socket (1272) may be configured to provide a snap-fit engagement with deflectable beams (1262) of plug (1260). While plug (1260) and socket (1272) are shown, it will be appreciated that cradle (1214) may be selectively coupled to cradle dock (1216) in any other suitable manner. For example, cradle (1214) may be selectively coupled to cradle dock (1216) via magnetic attraction between a first magnet (not shown) of cradle (1214) and a second magnet (not shown) of cradle dock (1216). In addition, or alternatively, cradle (1214) may be selectively coupled to cradle dock (1216) via hook-and-loop fasteners (not shown).

In the example shown, arch-shaped plate (1270) has a generally concave bottom (1274) that is contoured to complement an upper region of the operator's wrist, such that arch-shaped plate (1270) may fit snugly against the operator's wrist. To that end, cradle dock (1216) further includes a pair of opposed slots (1276) configured to receive wristband (1218), which may define a loop that is sized and configured to receive the operator's wrist to thereby secure fluid source assembly (1210) to the operator's wrist. In this manner, syringes (1212a, 1212b) may be operatively mounted to the operator's wrist and supported thereby via wristband (1218). In some versions, wristband (1218) may include quick-connect members in the form of first and second clasps (not shown), strips of hook-and-loop fastening features, or other features that are configured to selectively couple to each other for closing the loop defined by wristband (1218) and to selectively decouple from each other for opening the loop defined by wristband (1218), such as to assist the operator with donning and removing wristband (1218). In addition, or alternatively, wristband (1218) may comprise an elastomeric material to facilitate sliding of wristband (1218) over the operator's fist. Various other suitable ways in which wristband (1218) may be configured will be apparent to a person skilled in the art in view of the teachings herein.

In some versions, fluid source assembly (1210) may be secured to the wrist of the same hand with which the operator is grasping handle body (1120) of fluid injection instrument (1110). Such an arrangement may limit the potential movement of syringes (1212a, 1212b) relative to instrument (1110), such as in a direction away from instrument (1110); and may thereby minimize the risk of syringes (1212a, 1212b) tugging instrument (1110) via the respective supply tubes (1181a, 1181b). For example, such an arrangement may relieve instrument (1110) from bearing the weight of syringes (1212a, 1212b), as would be the case if syringes (1212a, 1212b) were simply suspended from instrument (1110) via supply tubes (1181, 1181b). In some versions, supply tubes (1181a, 1181b) may be provided with sufficient slack to permit some degree of movement of instrument (1110) relative to fluid source assembly (1210) while the operator grasps handle body (1120) with the same hand as that to which fluid source assembly (1210) is secured, such as to enable pivoting or other manipulation of instrument (1110) without syringes (1212a, 1212b) tugging instrument (1110) via the respective supply tubes (1181a, 1181b).

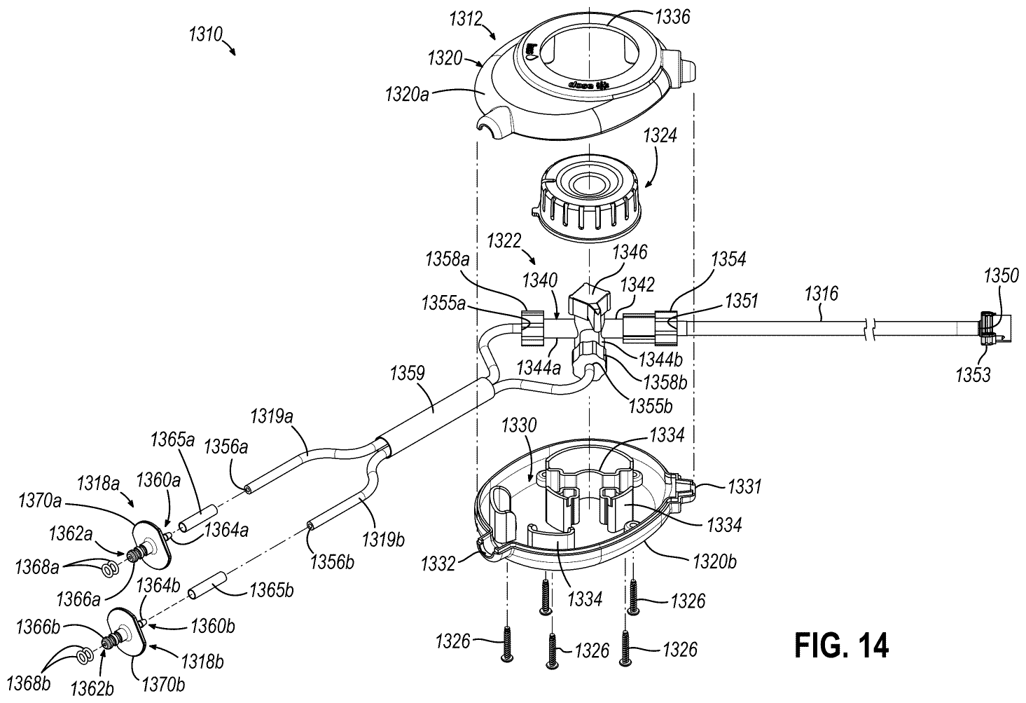

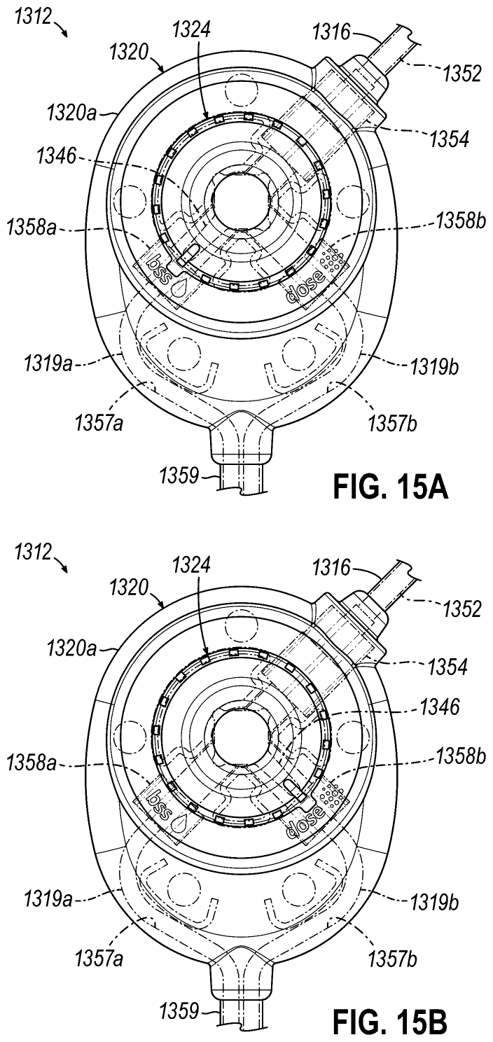

Referring now to FIGS. 14-15B, pressure control delivery assembly (1310) of the present example includes a fluid selection device (1312) that is coupled with a pressurized fluid medium source (1314) (FIG. 5) via a fluid medium supply tube (1316) and with first and second syringe adapters (1318a, 1318b) via first and second fluid medium delivery tubes (1319a, 1319b), respectively. Pressurized fluid medium source (1314) may contain any suitable pressurized fluid medium, such as air. Other suitable fluid media that may be used will be apparent to those skilled in the art in view of the teachings herein. Pressurized fluid medium source (1314) may be operatively coupled to a fluid pump (not shown) that is operable to pressurize the fluid medium. In some versions, pressurized fluid medium source (1314) may also be operatively coupled to a pressure regulator (not shown) that is operable to regular the fluid pressure of the pressurized fluid medium that is output from the pump. Various suitable forms that such a pump and/or regulator may take will be apparent to those skilled in the art in view of the teachings herein. By way of example only, such a pump and/or regulator may be configured and operable in accordance with at least some of the teachings of U.S. Pat. No. 10,258,502, entitled “Therapeutic Agent Delivery Device,” issued on Apr. 16, 2019, the disclosure of which is incorporated by reference herein.

Fluid selection device (1312) of the present example includes a generally puck-shaped rigid manifold (1320), a valve assembly (1322), and a selector knob (1324). In the example shown, manifold (1320) is formed from first and second shells (e.g., halves) (1320a, 1320b) fixedly coupled to each other along a longitudinal centerline of manifold (1320) via a plurality of fasteners in the form of screws (1326), such that shells (1320a, 1320b) collectively define manifold (1320). Shells (1320a, 1320b) may each be manufactured via 3D printing, injection molding, investment casting, machining, and/or any other suitable manufacturing techniques, and subsequently assembled to each other to form manifold (1320). It will be appreciated that manifold (1320) may be formed in any other suitable manner. For example, shells (1320a, 1320b) may be integrally formed together with each other as a unitary (e.g., monolithic) piece to define manifold (1320), such that the corresponding pairs of engagement features may be omitted.

As shown, manifold (1320) defines an interior cavity (1330) that is configured to at least partially house various components of valve assembly (1322), fluid medium supply tube (1316), fluid medium delivery tubes (1319a, 1319b), and selector knob (1324). In this regard, manifold (1320) of the present example also includes a proximal bore (1331) configured to receive a portion of fluid medium supply tube (1316) and a distal bore (1332) configured to receive portions of fluid medium delivery tubes (1319a, 1319b), such that fluid medium supply tube (1316) may extend out of interior cavity (1330) via proximal bore (1331) and fluid medium delivery tubes (1319a, 1319b) may extend out of interior cavity (1330) via distal bore (1332). In the example shown, a plurality of internal partitions (1334) extend partially across interior cavity (1330) and are spaced apart from each other to receive corresponding portions of valve assembly (1322) to thereby securely retain valve assembly (1322) within interior cavity (1330). Manifold (1320) of the present example further includes an upper bore (1336) configured to rotatably receive a lower portion of selector knob (1324).

Valve assembly (1322) of the present example is in the form of a three-way stopcock. Valve assembly (1322) of the present example includes a generally T-shaped valve body (1340) have an inlet port (1342) and first and second outlet ports (1344a, 1344b). Valve assembly (1322) also includes a flow selector (1346) pivotably coupled to valve body (1340) for selectively placing inlet port (1342) into and out of fluid communication with each of outlet ports (1344a, 1344b). In this regard, flow selector (1346) may be pivotable between a closed state (not shown) in which inlet port (1342) is fluidly isolated from each other outlet ports (1344a, 1344b), a first open state (FIG. 15A) in which inlet port (1342) is fluidly coupled with first outlet port (1344a) and fluidly isolated from second outlet port (1344b), and a second open state (FIG. 15B) in which inlet port (1342) is fluidly coupled with second outlet port (1344b) and fluidly isolated from first outlet port (1344a). Selector knob (1324) is fixedly coupled to flow selector (1346) such that the operator may pivot flow selector (1346) between the open and closed states via rotation of selector knob (1324).

In the example shown, fluid medium supply tube (1316) extends between an open proximal end (1350) and an open distal end (1351) and defines a lumen (1352) extending therebetween for directing air (or other fluid medium) therethrough. Open proximal end (1350) is fluidly connected to pressurized fluid medium source (1314) via a pneumatic fitting (1353), and open distal end (1351) is fluidly connected to inlet port (1342) via a pneumatic fitting (1354). In the example shown, fluid medium supply tube (1316) extends through proximal bore (1331) of manifold (1320), such that open distal end (1351) is positioned within interior cavity (1330) of manifold (1320) for coupling to inlet port (1342) while open proximal end (1350) is positioned external to manifold (1320) for coupling to pressurized fluid medium source (1314).

Each fluid medium delivery tube (1319a, 1319b) extends between an open proximal end (1355a, 1355b) and an open distal end (1356a, 1356b), and defines a lumen (1357a, 1357b) extending therebetween for directing fluid therethrough. Each open proximal end (1355a, 1355b) is fluidly connected to the respective outlet port (1344a, 1344b) via a corresponding pneumatic fitting (1358a, 1358b), and each open distal end (1356a, 1356b) is fluidly connected to the respective syringe adapter (1318a, 1318b), as described in greater detail below. In the example shown, fluid medium delivery tubes (1319a, 1319b) both extend through distal bore (1332) of manifold (1320), such that open proximal ends (1355a, 1355b) are positioned within interior cavity (1330) of manifold (1320) for coupling to the respective outlet ports (1344a, 1344b) while open distal ends (1356a, 1356b) are positioned external to manifold (1320) for coupling to the respective syringe adapters (1318a, 1318b). In the example shown, portions of fluid medium delivery tubes (1319a, 1319b) extend parallel to each other and are collectively wrapped in a braided sleeve (1359).

Each syringe adapter (1318a, 1318b) of this example has a proximal end (1360a, 1360b) and a distal end (1362a, 1362b). Each proximal end (1360a, 1360b) has a barbed connection feature (1364a, 1364b) that is adapted for connection to corresponding tubing (1365a, 1365b) which is, in turn, adapted for connection to the corresponding fluid medium delivery tube (1319a, 1319b). Each distal end (1362a, 1362b) comprises a tubular member (1366a, 1366b) having annular recesses (not shown) for receiving corresponding gaskets in the form of O-rings (1368a, 1368b). Each tubular member (1366a, 1366b) is sized and configured to be received in the second opening of the corresponding syringe barrel (1220a, 1220b), while each O-ring (1368a, 1368b) is configured to provide a fluid tight seal between the lumen of the corresponding syringe (1212a, 1212b) and the respective syringe adapter (1318a, 1318b) to prevent the escape of fluid pressure from the second opening of the corresponding syringe barrel (1220a, 1220b). A lumen (not shown) extends between each proximal end (1360a, 1360b) and each distal end (1362a, 1362b). A flange (1370a, 1370b) is disposed between proximal end (1360a, 1360b) and distal end (1362a, 1362b) of each syringe adapter (1318a, 1318b). In the present example, each syringe adapter (1318a, 1318b) is a single unitary body, but in other examples may comprise multiple portions coupled together. Various other suitable ways in which syringe adapters (1318a, 1318b) may be configured will be apparent to a person skilled in the art in view of the teachings herein. By way of example only, each syringe adapter (1318a, 1318b) may be configured and operable in accordance with at least some of the teachings of U.S. Pat. No. 10,258,502, entitled “Therapeutic Agent Delivery Device,” issued on Apr. 16, 2019, the disclosure of which is incorporated by reference herein.

As discussed in more detail below, proximal ends (1360a, 1360b) of syringe adapters (1318a, 1318b) may each be selectively coupled to pressurized fluid medium source (1314) and distal ends (1362a, 1362b) of syringe adapters (1318a, 1318b) may each be received in the second opening of the corresponding syringe (1212a, 1212b). Therefore, the pressurized air (or other fluid medium) may be selectively communicated to the lumen of first syringe (1212a) via first adapter (1318a), proximal to piston (1232a), and cause the advancement of piston (1232a) within first syringe (1212a) to thereby dispense fluid from first syringe (1212a). Likewise, the pressurized air (or other fluid medium) may be selectively communicated to the lumen of second syringe (1212b) via second adapter (1318b), proximal to piston (1232b), and cause the advancement of piston (1232b) within second syringe (1212b) to thereby dispense fluid from second syringe (1212b). In some instances, either adapter (1318a, 1318b) may be used to selectively communicate suction to the lumen of the corresponding syringe (1212a, 1212b), proximal to the respective piston (1232a, 1232b), and cause the retraction of the respective piston (1232a, 1232b) within the corresponding syringe (1212a, 1212b) to thereby draw fluid into the corresponding syringe (1212a, 1212b).

When flow selector (1346) of valve assembly (1322) is in the first open state shown in FIG. 15A, pressurized air (or other fluid medium) may flow from pressurized fluid medium source (1314), through lumen (1352) of fluid medium supply tube (1316), through inlet port (1342) and first outlet port (1344a) of valve body (1340), through lumen (1357a) of first fluid medium delivery tube (1319a), through the lumen of first adapter (1318a), and into the lumen of first syringe (1212a) for advancing piston (1232a) to thereby dispense bleb fluid (340) from first syringe (1212a) into first luer fitting (1180a). Bleb fluid (340) may then travel in a downstream direction along the flow path described above for delivery at beveled distal end (1144) of inner cannula (1135) of fluid injection instrument (1110). In this regard, the advancement of piston (1232a) may cause the fluid pressure within first proximal fluid supply passageway (1188a) of instrument (1110) to reach the aforementioned threshold fluid pressure such that first membrane (1199a) of first Belleville valve (1183a) may immediately bend toward the respective open state to permit flow of bleb fluid (340) in the downstream direction through first fluid gateway (1194a). Arrestment of such advancement of piston (1232a) may cause the fluid pressure within first proximal fluid supply passageway (1188a) of instrument (1110) to drop below the aforementioned threshold fluid pressure such that first membrane (1199a) of first Belleville valve (1183a) may immediately resiliently return to the respective closed state to halt the flow of bleb fluid (340) in the downstream direction through first fluid gateway (1194a) while also preventing backflow in the upstream direction.

When flow selector (1346) of valve assembly (1322) is in the second open state shown in FIG. 15B, pressurized air (or other fluid medium) may flow from pressurized fluid medium source (1314), through lumen (1352) of fluid medium supply tube (1316), through inlet port (1342) and second outlet port (1344b) of valve body (1340), through lumen (1357b) of second fluid medium delivery tube (1319b), through the lumen of second adapter (1318b), and into the lumen of second syringe (1212b) for advancing piston (1232b) to thereby dispense therapeutic fluid (341) from second syringe (1212b) into second luer fitting (1180b). Therapeutic fluid (341) may then travel in a downstream direction along the flow path described above for delivery at beveled distal end (1144) of inner cannula (1135) of fluid injection instrument (1110). In this regard, the advancement of piston (1232b) may cause the fluid pressure within second proximal fluid supply passageway (1188b) of instrument (1110) to reach the aforementioned threshold fluid pressure such that second membrane (1199b) of second Belleville valve (1183b) may immediately bend toward the respective open state to permit flow of therapeutic fluid (341) in the downstream direction through second fluid gateway (1194b). Arrestment of such advancement of piston (1232b) may cause the fluid pressure within second proximal fluid supply passageway (1188b) of instrument (1110) to drop below the aforementioned threshold fluid pressure such that second membrane (1199b) of second Belleville valve (1183b) may immediately resiliently return to the respective closed state to halt the flow of therapeutic fluid (341) in the downstream direction through second fluid gateway (1194b) while also preventing backflow in the upstream direction.

It will be appreciated that pressure control delivery assembly (1310) may enable rapid switching between advancement of pistons (1232a, 1232b) and thus between pumping of fluids (340, 341) to the corresponding fluid supply tubes (1181a, 1181b) for delivery at beveled distal end (1144) of inner cannula (1135). It will also be appreciated that fluid selection device (1312) of pressure control delivery assembly (1310) may be positioned remotely relative to syringes (1212a, 1212b). For example, fluid source assembly (1210) including syringes (1212a, 1212b) may be secured to the operator's wrist as described above, while fluid selection device (1312) may be positioned elsewhere (e.g., outside of the operator's reach) in the sterile environment in which the procedure is being performed, such as on a mayo stand or in the hands of a technician, with fluid medium delivery tubes (1319a, 1319b) and braided sleeve (1359) being sufficiently long to accommodate necessary movement of the operator relative to fluid selection device (1312). In this manner, the technician may manipulate selector knob (1324) for rapidly switching between pumping of fluids (340, 341) while the operator focuses on maintaining beveled distal end (1144) of inner cannula (1135) in the subretinal space. In other words, such remote positioning of fluid selection device (1312) relative to syringes (1212a, 1212b) may enable someone other than the operator to control the delivery of fluids (340, 341) at beveled distal end (1144). Nevertheless, in some scenarios, fluid selection device (1312) may be positioned within the operator's reach such that the operator may control the delivery of fluids (340, 341) at beveled distal end (1144). For example, fluid selection device (1312) may be secured to the operator (e.g., to the same wrist to which fluid source assembly (1210) is secured or along the operator's forearm), in a manner similar to that described above with respect to fluid source assembly (1210). In some such cases, fluid medium delivery tubes (1319a, 1319b) and braided sleeve (1359) may be relatively short in length (e.g., less than or equal to a length of the operator's forearm), while fluid medium supply tube (1316) may be sufficiently long to accommodate necessary movement of the operator relative to pressurized fluid medium source (1314).

It should therefore be understood that system (1000) including fluid injection instrument (1110), fluid source assembly (1210), and pressure control delivery assembly (1310) may be used to perform the subretinal delivery of bleb fluid (340) and therapeutic agent (341) described above with respect to FIGS. 3-4J. Other suitable ways in which system (1000) including fluid injection instrument (1110), fluid source assembly (1210), and/or pressure control delivery assembly (1310) may be used will be apparent to those skilled in the art in view of the teachings herein.

### B. Second Example of Transvitreal Subretinal Injection System

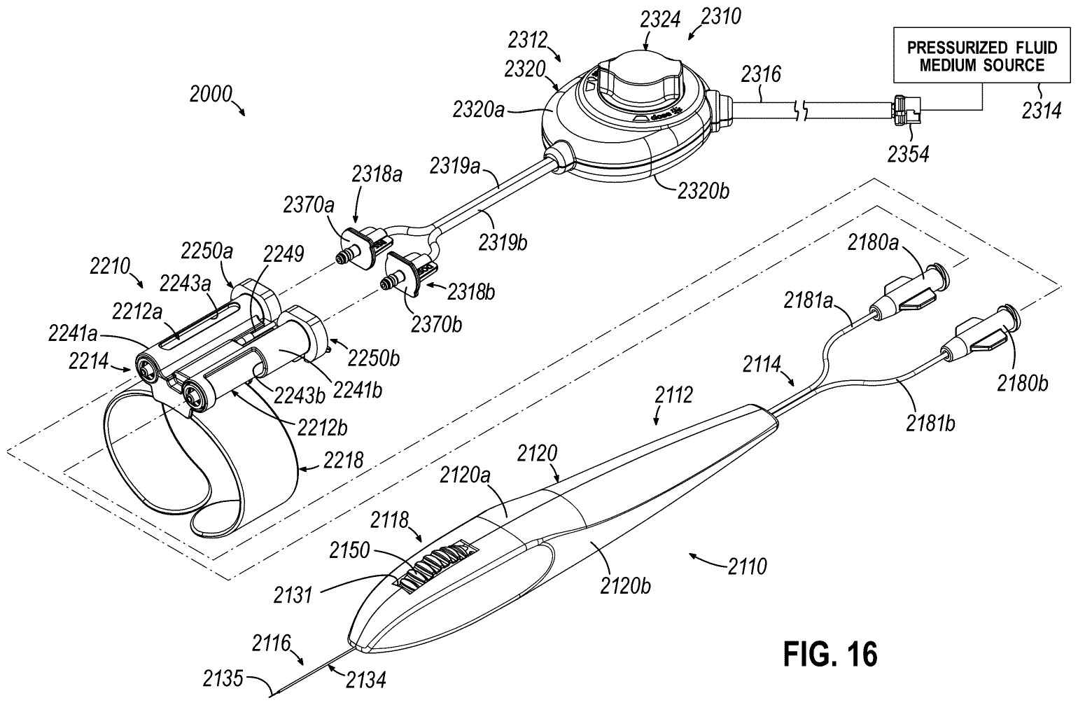

FIG. 16 shows an example of a system (2000) that comprises a fluid injection instrument (2110), a fluid source assembly (2210), and a pressure control delivery assembly (2310) for delivering one or more fluids during a procedure to treat an ocular condition, such as the subretinal delivery of therapeutic agent (341) described above. System (2000) may be configured and operable like system (1000) described above, except as otherwise described below. While fluid injection instrument (2110), fluid source assembly (2210), and pressure control delivery assembly (2310) are described below in connection with system (2000), it will be appreciated that any of fluid injection instrument (2110), fluid source assembly (2210), and/or pressure control delivery assembly (2310) may be readily incorporated into system (1000) described above. For example, fluid injection instrument (2110) may be readily incorporated into system (1000) in place of fluid injection instrument (1110); fluid source assembly (2210) may be readily incorporated into system (1000) in place of fluid source assembly (1210); and/or pressure control delivery assembly (2310) may be readily incorporated into system (1000) in place of pressure control delivery assembly (1310).

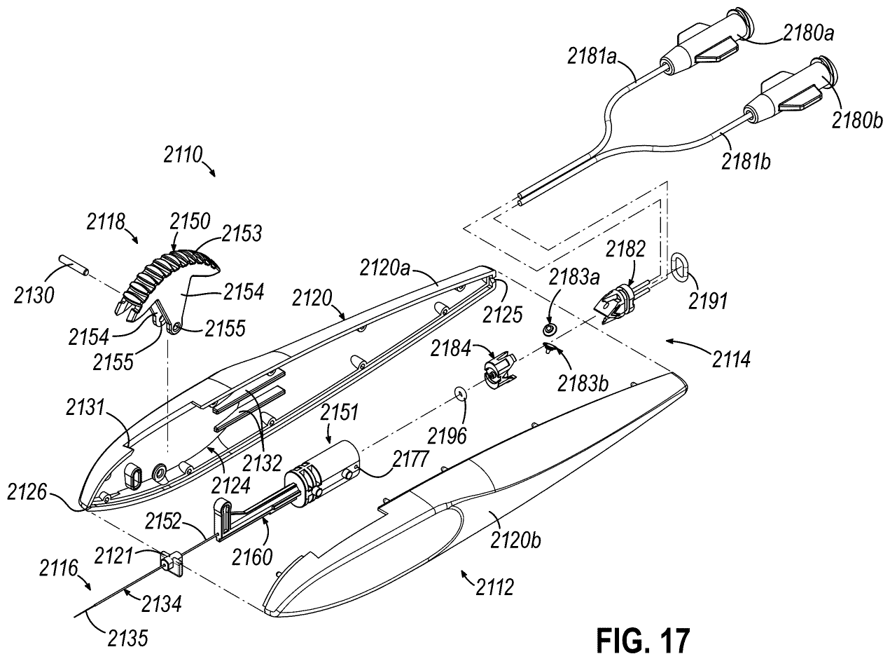

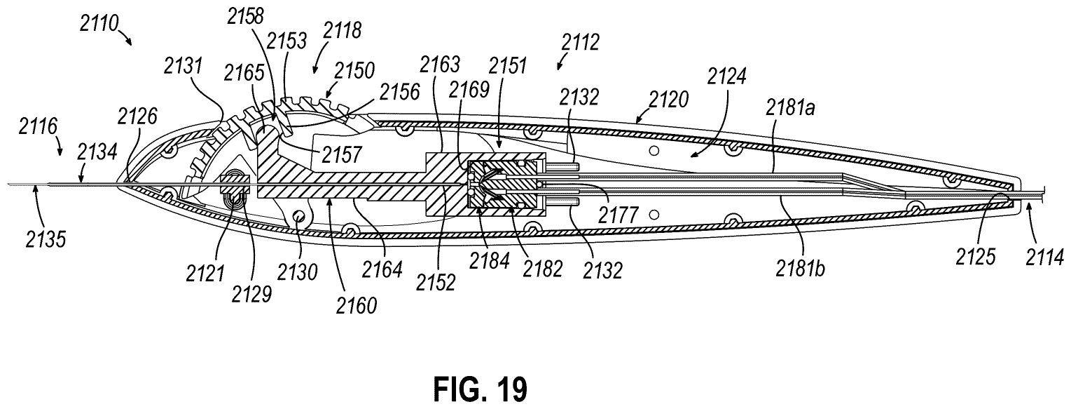

Referring primarily to FIGS. 17-19, with continuing reference to FIG. 16, fluid injection instrument (2110) may be configured and operable like instrument (1110) described above, except as otherwise described below. In this regard, fluid injection instrument (2110) of the present example includes a handle assembly (2112), a fluid supply assembly (2114), a fluid delivery assembly (2116), and an actuation assembly (2118).

Handle assembly (2112) of this example includes a rigid handle body (2120) and a cannula hub (2121). In some versions, handle assembly (2112) may further include a strain relief (not shown) similar to strain relief (1122). In the example shown, handle body (2120) is formed from first and second shells (e.g., halves) (2120a, 2120b) fixedly coupled to each other along a longitudinal centerline of handle body (2120) via corresponding pairs of engagement features (e.g., interlocking detents and indents), such that shells (2120a, 2120b) collectively define handle body (2120).

As shown, handle body (2120) defines an interior cavity (2124) that is configured to at least partially house various components of fluid supply assembly (2114), fluid delivery assembly (2116), and actuation assembly (2118). In this regard, handle body (2120) of the present example also includes a proximal bore (2125) configured to receive a portion of fluid supply assembly (2114) and a distal bore (2126) configured to receive a portion of fluid delivery assembly (2116), such that fluid supply assembly (2114) may extend out of interior cavity (2124) via proximal bore (2125) and fluid delivery assembly (2116) may extend out of interior cavity (2124) via distal bore (2126). In the example shown, cannula hub (2121) is fixedly coupled to handle body (2120) within interior cavity (2124) and includes a central bore (2129) configured to receive a portion of fluid delivery assembly (2116) such that cannula hub (2121) may provide support to fluid delivery assembly (2116) within interior cavity (2124).

Handle body (2120) of the present example also includes a pivot pin (2130) extending laterally across interior cavity (2124) and configured to be pivotably received by corresponding portions of actuation assembly (2118), as well as an upper slot (2131) positioned generally above pivot pin (2130) and configured to expose a corresponding portion of actuation assembly (2118) to an exterior of handle body (2120), as described in greater detail below. In the example shown, handle body (2120) further includes a plurality of (e.g., four) longitudinal rails (2132) (two shown) configured to slidably support corresponding portions of actuation assembly (2118).

Fluid delivery assembly (2116) of the present example includes an outer cannula (2134) and an inner cannula (2135) that are similar to outer cannula (1134) and inner cannula (1135) described above, respectively.

Actuation assembly (2118) of the present example includes a pivotable scroll wheel (2150), a translatable cannula sled (2151), and a translatable cannula support tube (2152) configured to cooperate with each other to drive translation of inner cannula (2135) relative to outer cannula (2134). In the example shown, scroll wheel (2150) is pivotably coupled to handle body (2120) and includes a generally arch-shaped grip portion (2153) and a pair of laterally-opposed arms (2154) extending generally downwardly from grip portion (2153). Scroll wheel (2150) of the present example also includes a pair of pivot bores (2155) each extending laterally through a lower portion of a respective arm (2154) and configured to pivotably receive pivot pin (2130) of handle body (2120) for pivotably coupling scroll wheel (2150) to handle body (2120), such that scroll wheel (2150) may be pivotable about a pivot axis defined by pivot pin (2130) and pivot bores (2155) between an unactuated state and at least one actuated state, such as a fully actuated state. In this regard, grip portion (2153) may be at least partially exposed to an exterior of handle body (2120) via upper slot (2131) to enable the operator to access and manipulate grip portion (2153) between the unactuated and fully actuated states with the operator's thumb, for example.

In the example shown, scroll wheel (2150) also includes a pair of parallel flanges (2156) extending generally downwardly from grip portion (2153) between arms (2154) and having confronting, generally planar cam surfaces (2157) that are spaced apart from each other to define a socket (2158) that is configured to receive a portion of cannula sled (2151).

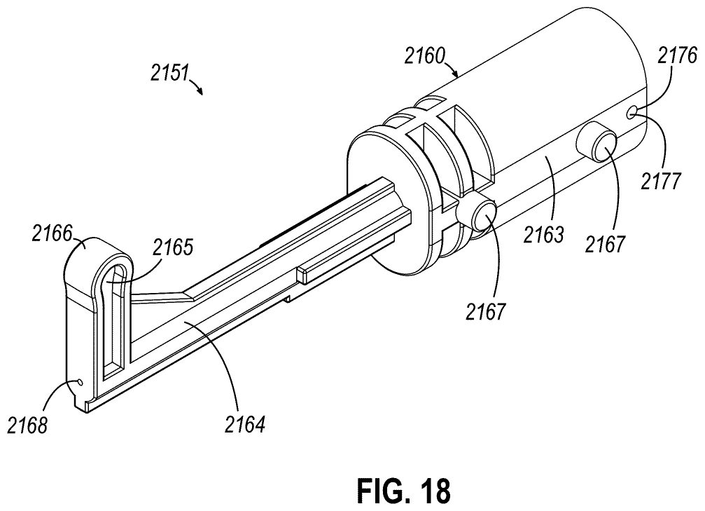

In this regard, cannula sled (2151) of the present example is translatably coupled to handle body (2120) and includes a sled housing (2160). As best shown in FIG. 18, sled housing (2160) includes a drum (2163) and a yoke (2164) extending distally from drum (2163) and having a generally bulbous distal hitch (2165) extending generally upwardly for receipt within socket (2158) of scroll wheel (2150). In the example shown, hitch (2165) has a generally C-shaped cam surface (2166) configured to be cammingly engaged by one or both planar cam surfaces (2157) of scroll wheel (2150) to convert pivoting of scroll wheel (2150) into translation of cannula sled (2151), such that cannula sled (2151) may be longitudinally translatable between a proximal position and a distal position in response to pivoting of scroll wheel (2150) between the unactuated and fully actuated states. Drum (2163) of the present example includes a plurality of (e.g., four) lateral projections (2167) (two shown) configured to be slidably received between corresponding pairs of longitudinal rails (2132) of handle body (2120), such that the interaction between rails (2132) and projections (2167) may guide longitudinal translation of cannula sled (2151) between the proximal and distal positions during pivoting of scroll wheel (2150) between the unactuated and fully actuated states.

As shown, yoke (2164) of sled housing (2160) also includes a central bore (2168) configured to securely receive cannula support tube (2152) to fix cannula support tube (2152) against movement relative to sled housing (2160), such that cannula support tube (2152) may be longitudinally translatable together with cannula sled (2151). In this regard, central bore (2168) has an inner cross-dimension (e.g., diameter) substantially equal to an outer cross-dimension (e.g., diameter) of cannula support tube (2152) to provide a friction fit therebetween. In the example shown, a proximal end of outer cannula (2134) is disposed distal of central bore (2168). Drum (2163) of sled housing (2160) also includes a chamber (2169) disposed proximal of central bore (2168) and configured to securely retain at least a portion of fluid supply assembly (2114). In this regard, drum (2163) includes a pair of laterally-opposed bores (2176) (one shown) configured to selectively receive a retention pin (2177) for retaining various components of fluid supply assembly (2114) within chamber (2169).

In the example shown, cannula support tube (2152) is slidably received within outer cannula (2134); and is configured to securely receive inner cannula (2135) to fix inner cannula (2135) against movement relative to cannula support tube (2152) (and thus relative to connection member (2162)), such that inner cannula (2135) may be longitudinally translatable together with cannula support tube (2152) and cannula sled (2151).

Fluid supply assembly (2114) of the present example includes first and second luer fittings (2180a, 2180b), first and second fluid supply tubes (2181a, 2181b), a fluid supply junction (2182), first and second self-actuating check valves (2183a, 2183b), a valve retainer (2184), a generally obround O-ring (2191), and a generally circular O-ring (2196) that are similar to first and second luer fittings (1180a, 1180b), first and second fluid supply tubes (1181a, 1181b), fluid supply junction (1182), first and second self-actuating check valves (1183a, 1183b), valve retainer (1184), generally obround O-ring (1191), and generally circular O-ring (1196) described above, respectively.

As shown, fluid supply junction (2182) and valve retainer (2184) are each securely retained within chamber (2169) of sled housing (2160) of actuation assembly (2118). More particularly, retention pin (2177) (FIG. 17) captures fluid supply junction (2182) and valve retainer (2184) within chamber (2169). O-ring (2191) provides a fluid-tight seal between fluid supply junction (2182) and a side of chamber (2169), while O-ring (2196) provides a fluid-tight seal between valve retainer (2184) and a distal end of chamber (2169). Thus, rather than being housed within a connection member that is securely retained within chamber (2169) of sled housing (2160), various components of fluid supply assembly (2114) may be securely retained directly within chamber (2169) of sled housing (2160).

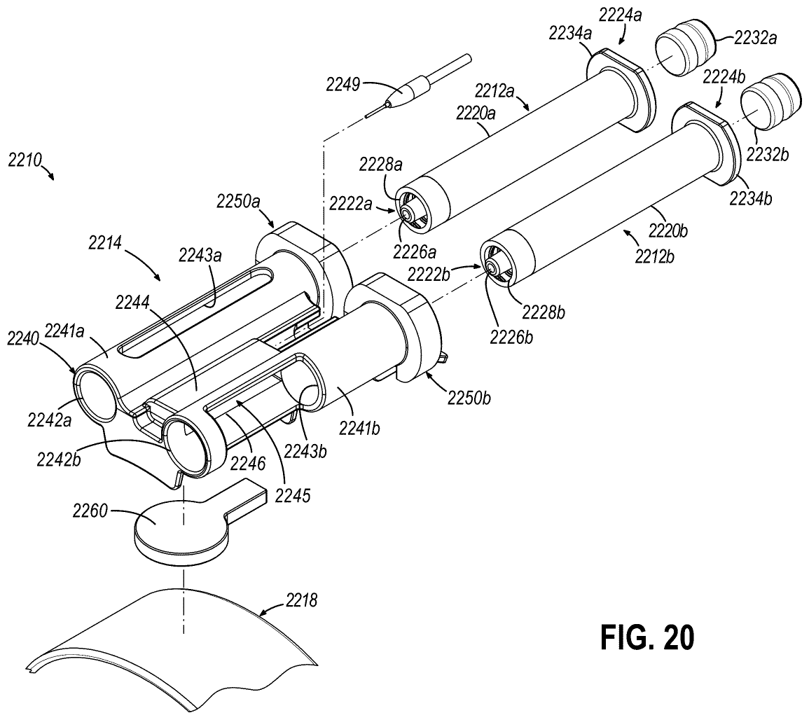

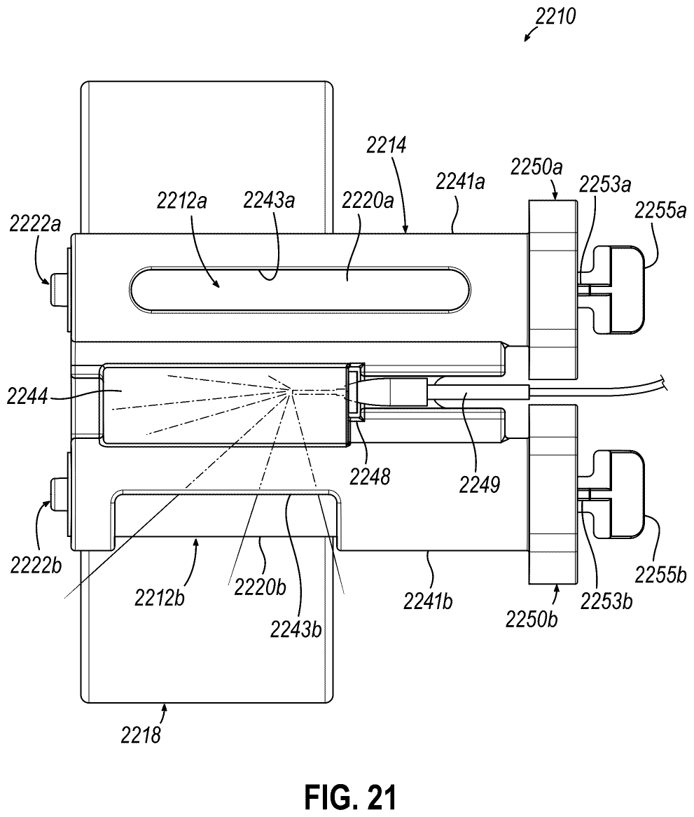

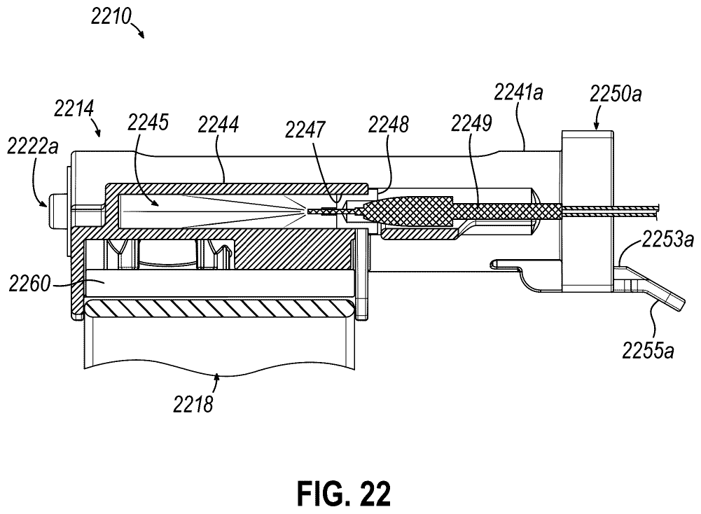

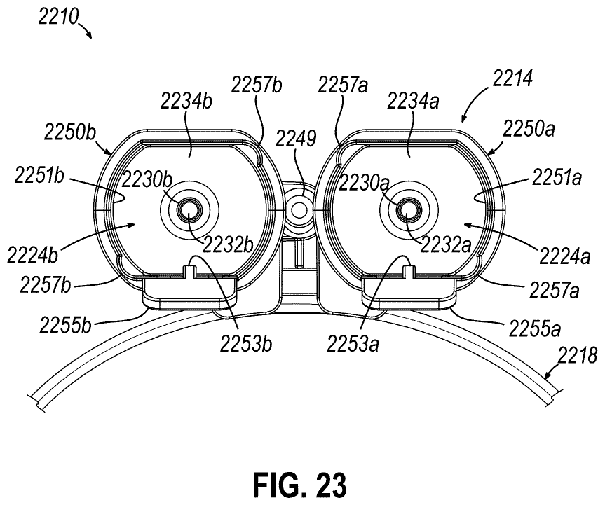

Referring now to FIGS. 20-23, fluid source assembly (2210) may be configured and operable like fluid source assembly (1210) described above, except as otherwise described below. In this regard, fluid source assembly (2210) of the present example includes first and second fluid sources in the form of syringes (2212a, 2212b), a syringe cradle (2214), and a wearable component in the form of a magnetic cuff (2218). As shown, each syringe (2212a, 2212b) includes a barrel (2220a, 2220b) having a distal end (2222a, 2222b), a proximal end (2224a, 2224b), and a lumen (not shown) extending therebetween. Each distal end (2222a, 2222b) includes a first, dispensing opening (2226a, 2226b) and a threaded portion (2228a, 2228b) that enables coupling of the respective syringe (2212a, 2212b) to a needle, tubing, etc. In the example shown, threaded portions (2228a, 2228b) are configured to threadably engage first and second luer fittings (2180a, 2180b), respectively, for fluidly coupling the respective lumens of syringes (2212a, 2212b) with corresponding lumens of supply tubes (2181a, 2181b). In this regard, the lumen of first syringe (2220a) contains bleb fluid (340) while the lumen of second syringe (2220b) contains therapeutic fluid (341). Each proximal end (2224a, 2224b) includes a second opening (2230a, 2230b) that is configured to receive a corresponding plunger piston (2232a, 2232b). Proximal end (2224a, 2224b) of each syringe barrel (2220a, 2220b) comprises a flange (2234a, 2234b) that extends radially outwardly relative to a longitudinal axis of the respective syringe (2212a, 2212b) and that may act as a finger grip when a user holds the respective syringe (2212a, 2212b), for example.

Syringe cradle (2214) of the present example includes a cradle body (2240) having a pair of parallel, longitudinal sheaths (2241a, 2241b) that define respective bores (2242a, 2242b) configured to selectively retain respective syringe barrels (2220a, 2220b) for selectively securing syringes (2212a, 2212b) to syringe cradle (2214). In this regard, each bore (2242a, 2242b) may have an inner cross-dimension (e.g., diameter) at least slightly greater than an outer cross-dimension (e.g., diameter) of the respective syringe barrel (2220a, 2220b). In the example shown, each sheath (2241a, 2241b) includes a respective elongate slot (2243a, 2243b) for enabling visual observation of the respective syringe barrel (2220a, 2220b) therethrough. More particularly, an upper elongate slot (2243a) extends through a top of first sheath (2241a) for enabling visual observation of first syringe barrel (2220a) therethrough, and a lateral elongate slot (2243b) extends through a laterally outer side of second sheath (2241b) for enabling visual observation of second syringe barrel (2220b) therethrough.

In this regard, cradle body (2240) of the present example further has a central body portion (2244) extending between sheaths (2241a, 2241b) and defining an illumination chamber (2245) that opens into second bore (2242b) via a lateral aperture (2246). A chandelier port (2247) extends through a proximal wall of central body portion (2244) to illumination chamber (2245) and securely retains a chandelier socket (2248), which is sized and configured to selectively receive a chandelier, such as a fiber optic chandelier (2249), to allow an illuminating end of chandelier (2249) to be disposed within and/or illuminate illumination chamber (2245). It will be appreciated that light emitted by chandelier (2249) may pass from illumination chamber (2245) into second bore (2242b) through lateral aperture (2246), and may thereby illuminate second syringe barrel (2220b). Such illumination of second syringe barrel (2220b) may be visually observed through lateral elongate slot (2243b). In this manner, chandelier light (2249) may be configured to provide backlighting of second syringe barrel (2220b), which may assist the operator with determining an amount of therapeutic agent (341) remaining in the lumen of second syringe (2220b) and/or an amount of therapeutic agent (341) that has been dispensed from first opening (2226b) of second syringe (2220b). While chandelier socket (2248) has been described for selectively receiving chandelier (2249), chandelier (2249) may be permanently coupled to cradle body (2240) with the illuminating end of chandelier (2249) permanently disposed within illumination chamber (2245) in some other versions. In addition, or alternatively, chandelier (2249) may include a light source, such as a light emitting diode (LED), disposed within illumination chamber (2245).

In the example shown, a pair of collars (2250a, 2250b) are positioned at a proximal end of cradle body (2240) in alignment with respective bores (2242a, 2242b). Collars (2250a, 2250b) enable the respective syringes (2212a, 2212b) to be coupled with a system with one or more powered components as described in greater detail below. Each collar (2250a, 2250b) may be used to secure corresponding portions of pressure control delivery assembly (2310) (e.g., syringe adapters (2318a, 2318b) described below) to proximal ends (2224a, 2224b) of syringes (2212a, 2212b) and thereby prevent movement of such portions of pressure control delivery assembly (2310) relative to the respective syringes (2212a, 2212b). In this regard, each collar (2250a, 2250b) defines a respective proximally-facing recess (2251a, 2251b) that is sized and configured to receive at least a portion of the flange (2234a, 2234b) of the respective syringe (2212a, 2212b) and/or a corresponding portion of pressure control delivery assembly (2310) (e.g., a corresponding flange (2370a, 2370b) described below). In the example shown, a pair of retention tabs (2253a, 2253b) are configured to sandwich the respective portions of pressure control delivery assembly (2310) against the flange (2234a, 2234b) of the respective syringe (2212a, 2212b) within the recess (2251a, 2251b) of the respective collar (2250a, 2250b). In the example shown, retention tabs (2253a, 2253b) are provided on respective deflectable beams (2255a, 2255b) extending along the bottoms of the respective collars (2250a, 2250b). In other versions, deflectable beams (2255a, 2255b) may extend along the sides (e.g., the laterally outer sides) of the respective collars (2250a, 2250b). Each recess (2251a, 2251b) of the present example includes at least one corresponding keyway (2257a, 2257b), with the at least one keyway (2257a) of first recess (2251a) having a unique configuration (e.g., size, shape, and/or angular position) relative to the at least one keyway (2257b) of second recess (2251b), the purposes of which are described below.

As shown, a coupling member in the form of a magnet (2260) is provided on an underside of cradle body (1240) that provides magnetic attraction to magnetic cuff (2218) for facilitating selective coupling and removal of cradle (2214) to and from magnetic cuff (2218). In this regard, magnetic cuff (2218) of the present example includes one or more ferrous elements (e.g., ferrous metal filings embedded in the cuff material, a single thin metallic sheet embedded in the cuff material, etc.) that provide magnetic attraction with magnet (2260). In some other versions, magnetic cuff (2218) includes an array of magnetic elements that provide magnetic attraction with magnet (2260). Various suitable features and configurations that may be incorporated into magnetic cuff (2218) to provide magnetic attraction with magnet (2260) will be apparent to those of ordinary skill in the art in view of the teachings herein. Magnetic cuff (2218) may define a loop that is sized and configured to receive the operator's wrist to thereby secure fluid source assembly (2210) to the operator's wrist. In this manner, syringes (2212a, 2212b) may be operatively mounted to the operator's wrist and supported thereby via magnetic cuff (2218). In some versions, magnetic cuff (2218) may include quick-connect members in the form of first and second clasps (not shown), strips of hook-and-loop fastening features, or other features that are configured to selectively couple to each other for closing the loop defined by magnetic cuff (2218) and to selectively decouple from each other for opening the loop defined by magnetic cuff (2218), such as to assist the operator with donning and removing magnetic cuff (2218). In addition, or alternatively, magnetic cuff (2218) may comprise an elastomeric material to facilitate sliding of magnetic cuff (2218) over the operator's fist. Various other suitable ways in which magnetic cuff (2218) may be configured will be apparent to a person skilled in the art in view of the teachings herein.

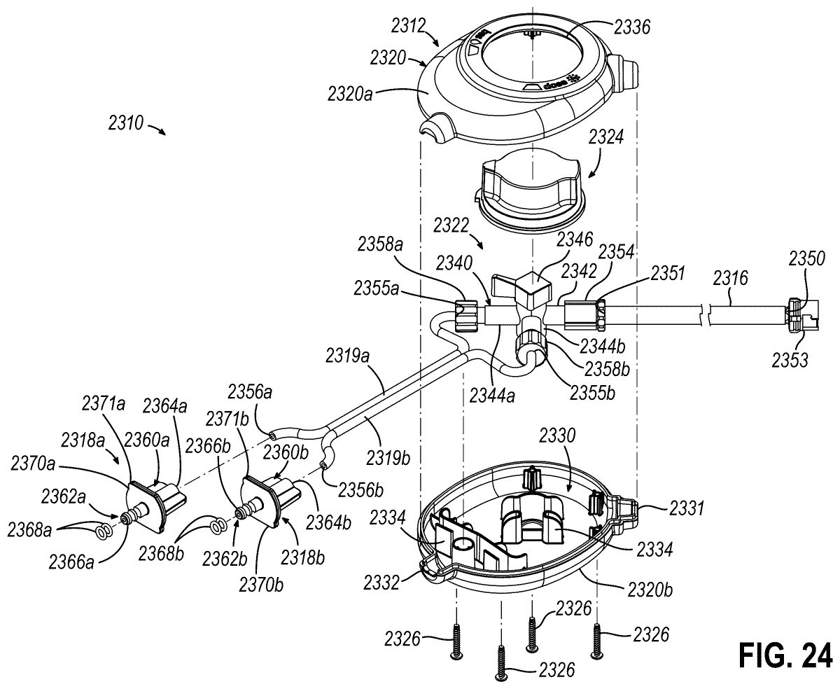

Referring now to FIG. 24, pressure control delivery assembly (2310) may be similar to pressure control delivery assembly (1310) described above, except as otherwise described below. In this regard, pressure control delivery assembly (2310) of the present example includes a fluid selection device (2312) that is coupled with a pressurized fluid medium source (2314) (FIG. 16) via a fluid medium supply tube (2316) and with first and second syringe adapters (2318a, 2318b) via first and second fluid medium delivery tubes (2319a, 2319b), respectively.

Fluid selection device (2312) of the present example includes a generally puck-shaped rigid manifold (2320), a valve assembly (2322), and a selector knob (2324). In the example shown, manifold (2320) is formed from first and second shells (e.g., halves) (2320a, 2320b) fixedly coupled to each other along a longitudinal centerline of manifold (2320) via a plurality of fasteners in the form of screws (2326), such that shells (2320a, 2320b) collectively define manifold (2320).

As shown, manifold (2320) defines an interior cavity (2330) that is configured to at least partially house various components of valve assembly (2322), fluid medium supply tube (2316), fluid medium delivery tubes (2319a, 2319b), and selector knob (2324). In this regard, manifold (2320) of the present example also includes a proximal bore (2331) configured to receive a portion of fluid medium supply tube (2316) and a distal bore (2332) configured to receive portions of fluid medium delivery tubes (2319a, 2319b), such that fluid medium supply tube (2316) may extend out of interior cavity (2330) via proximal bore (2331) and fluid medium delivery tubes (2319a, 1319b) may extend out of interior cavity (2330) via distal bore (2332). In the example shown, a plurality of internal partitions (2334) extend partially across interior cavity (2330) and are spaced apart from each other to receive corresponding portions of valve assembly (2322) to thereby securely retain valve assembly (2322) within interior cavity (2330). Manifold (2320) of the present example further includes an upper bore (2336) configured to rotatably receive a lower portion of selector knob (2324).

Valve assembly (2322) of the present example is in the form of a three-way stopcock. Valve assembly (2322) of the present example includes a generally T-shaped valve body (2340) have an inlet port (2342) and first and second outlet ports (2344a, 2344b). Valve assembly (2322) also includes a flow selector (2346) pivotably coupled to valve body (2340) for selectively placing inlet port (2342) into and out of fluid communication with each of outlet ports (2344a, 2344b) in a manner similar to that described above in connection with FIGS. 15A-15B.

In the example shown, fluid medium supply tube (2316) extends between an open proximal end (2350) and an open distal end (2351) and defines a lumen (2352) extending therebetween for directing air (or other fluid medium) therethrough. Open proximal end (2350) is fluidly connected to pressurized fluid medium source (2314) via a pneumatic fitting (2353), and open distal end (2351) is fluidly connected to inlet port (2342) via a pneumatic fitting (2354).

Each fluid medium delivery tube (2319a, 1319b) extends between an open proximal end (2355a, 1355b) and an open distal end (2356a, 1356b), and defines a lumen (2357a, 1357b) extending therebetween for directing fluid therethrough. Each open proximal end (2355a, 1355b) is fluidly connected to the respective outlet port (2344a, 1344b) via a corresponding pneumatic fitting (2358a, 1358b), and each open distal end (2356a, 1356b) is fluidly connected to the respective syringe adapter (2318a, 1318b), as described in greater detail below.

Each syringe adapter (2318a, 2318b) of this example has a proximal end (2360a, 2360b) and a distal end (2362a, 2362b). Each proximal end (2360a, 2360b) has a hollow connection feature (2364a, 2364b) that is adapted for connection to the corresponding fluid medium delivery tube (2319a, 2319b). Each distal end (2362a, 2362b) comprises a tubular member (2366a, 2366b) having annular recesses (not shown) for receiving corresponding gaskets in the form of O-rings (2368a, 2368b). Each tubular member (2366a, 2366b) is sized and configured to be received in the second opening of the corresponding syringe barrel (2220a, 2220b), while each O-ring (2368a, 2368b) is configured to provide a fluid tight seal between the lumen of the corresponding syringe (2212a, 2212b) and the respective syringe adapter (2318a, 2318b) to prevent the escape of fluid pressure from the second opening of the corresponding syringe barrel (2220a, 2220b). A lumen (not shown) extends between each proximal end (2360a, 2360b) and each distal end (2362a, 2362b). A flange (2370a, 2370b) is disposed between proximal end (2360a, 2360b) and distal end (2362a, 2362b) of each syringe adapter (2318a, 2318b).

As discussed in more detail above in connection with FIGS. 14-15B, proximal ends (2360a, 2360b) of syringe adapters (2318a, 2318b) may each be selectively coupled to pressurized fluid medium source (2314) and distal ends (2362a, 2362b) of syringe adapters (2318a, 2318b) may each be received in the second opening of the corresponding syringe (2212a, 2212b). In this regard, each flange (2370a, 2370b) of the present example includes at least one corresponding key (2371a, 2371b) configured to be received within corresponding keyways (2257a, 2257b) of recesses (2251a, 2251b), with the at least one key (2371a) of first flange (2370a) having a unique configuration (e.g., size, shape, and/or angular position) relative to the at least one key (2371b) of second flange (2370b). More particularly, the at least one key (2371a) of first flange (2370a) is configured to be received within the at least one keyway (2257a) of first recess (2251a) but not within the at least one keyway (2257b) of second recess (2251b); and the at least one key (2371b) of second flange (2370b) is configured to be received within the at least one keyway (2257b) of second recess (2251b) but not within the at least one keyway (2257a) of first recess (2251a). In this manner, keys (2371a, 2371b) of flanges (2370a, 2370b) and keyways (2257a, 2257b) of recesses (2251a, 2251b) may cooperate with each other to inhibit distal ends (2362a, 2362b) of syringe adapters (2318a, 2318b) from being inadvertently received in the second opening of the incorrect syringe (2212a, 2212b), thereby ensuring that the pressurized air (or other fluid medium) is selectively communicated to the lumen of first syringe (2212a) via first adapter (2318a), and that the pressurized air (or other fluid medium) is selectively communicated to the lumen of second syringe (2212b) via second adapter (2318b).

It should therefore be understood that system (2000) including fluid injection instrument (2110), fluid source assembly (2210), and pressure control delivery assembly (2310) may be used to perform the subretinal delivery of bleb fluid (340) and therapeutic agent (341) described above with respect to FIGS. 3-4J. Other suitable ways in which system (2000) including fluid injection instrument (2110), fluid source assembly (2210), and/or pressure control delivery assembly (2310) may be used will be apparent to those skilled in the art in view of the teachings herein.

While systems (1000, 2000) including fluid injection instruments (1110, 2110), fluid source assembly (1210, 2210), and pressure control delivery assembly (1310, 2310) have been described in the context of delivery of bleb fluid (340) and therapeutic agent (341) to the subretinal space from a transvitreal, trans-retinal approach, it will be appreciated that various components of either system (1000, 2000) may be used for delivery of bleb fluid (340) and/or therapeutic agent (341) to the subretinal space from any other suitable type of approach. In some versions, either fluid supply assembly (1114, 2114) may be readily incorporated into a fluid injection instrument that is configured to deliver bleb fluid (340) and/or therapeutic agent (341) to the subretinal space from a suprachoroidal approach, such as for selectively fluidly coupling first and second conduits to a needle of such a fluid injection instrument to supply bleb fluid (340) and therapeutic agent (341), respectively, to the needle. For example, either fluid supply assembly (1114, 2114) may be readily incorporated into a fluid injection instrument that is configured and operable in accordance with at least some of the teachings of U.S. Pat. No. 10,646,374, entitled “Apparatus and Method to Form Entry Bleb for Subretinal Delivery of Therapeutic Agent,” issued on May 12, 2020, the disclosure of which is incorporated by reference herein.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a body; (b) an outer cannula extending distally from the body along a longitudinal axis, wherein the outer cannula is fixedly secured to the body, wherein the outer cannula includes: (i) a proximal portion having a first cross-dimension, and (ii) a distal portion having at least one second cross-dimension smaller than the first cross-dimension, wherein the distal portion is configured to guide the cylindrical proximal portion into a trocar port for advancing the outer cannula into an eye of a patient; (c) an inner cannula extending along the longitudinal axis and slidably disposed in the outer cannula for translating longitudinally relative to the outer cannula, wherein the inner cannula includes: (i) an open proximal end, (ii) an open distal end configured to pierce a retina of the eye, and (iii) a lumen extending between the open proximal end and the open distal end, wherein the lumen is configured to communicate at least one fluid from the open proximal end to the open distal end for delivering the at least one fluid into a subretinal region of the eye.

### Example 2

The apparatus of Example 1, wherein the distal portion tapers radially inwardly in a distal direction.

### Example 3

The apparatus of Example 1, wherein the distal portion curves radially inwardly in a distal direction.

### Example 4

The apparatus of any of Examples 1 through 3, wherein the proximal portion is generally cylindrical.

### Example 5

The apparatus of Example 4, wherein the distal portion is generally frustoconical.

### Example 6

The apparatus of any of Examples 1 through 5, wherein the proximal portion defines a proximal opening, wherein the distal portion defines a distal opening smaller than the proximal opening.

### Example 7

The apparatus of Example 6, wherein the distal opening is configured to maintain centration of the inner cannula relative to the outer cannula during longitudinal translation of the inner cannula relative to the outer cannula.

### Example 8

The apparatus of any of Examples 1 through 7, wherein the open distal end is beveled.

### Example 9

The apparatus of Example 8, wherein the open distal end is oriented relative to the longitudinal axis at an angle of about 45 degrees.

### Example 10

The apparatus of any of Examples 1 through 9, wherein the outer cannula is rigid.

### Example 11

The apparatus of any of Examples 1 through 10, wherein the outer cannula comprises a metallic material.

### Example 12

The apparatus of Example 11, wherein the outer cannula comprises surgical stainless steel.

### Example 13

The apparatus of any of Examples 1 through 12, wherein the inner cannula is flexible.

### Example 14

The apparatus of any of Examples 1 through 13, wherein the inner cannula comprises a polymeric material.

### Example 15

The apparatus of Example 14, wherein the inner cannula comprises polyimide.

### Example 16

The apparatus of any of Examples 1 through 15, the at least one fluid including first and second fluids, the apparatus further comprising first and second proximal fluid supply passageways configured to be selectively fluidly coupled to the lumen of the inner cannula for supplying the first and second fluids, respectively, to the lumen.

### Example 17

The apparatus of Example 16, further comprising first and second valves configured to selectively fluidly couple the first and second proximal fluid supply passageways, respectively, to the lumen of the inner cannula.

### Example 18

The apparatus of Example 17, wherein the first and second valves include first and second Belleville valves, respectively.

### Example 19

The apparatus of any of Examples 17 through 18, wherein the first and second valves are each configured to fluidly couple the respective first or second proximal fluid supply passageway to the lumen of the inner cannula in response to a fluid pressure within the respective first or second fluid passageway being equal to or greater than a threshold fluid pressure.

### Example 20

The apparatus of Example 19, wherein the first and second valves are each configured to fluidly isolate the respective first or second proximal fluid supply passageway from the lumen of the inner cannula in response to a fluid pressure within the respective first or second fluid passageway being less than the threshold fluid pressure.

### Example 21

An apparatus, comprising: (a) a wearable component configured to be worn by an operator; and (b) at least one syringe supported by the wearable component, wherein the at least one syringe is configured to dispense at least one fluid to a fluid injection instrument held by the operator.

### Example 22

The apparatus of Example 21, wherein the wearable component includes at least one of a wristband or a cuff configured to be secured to a wrist of the operator.

### Example 23

The apparatus of any of Examples 21 through 22, wherein the at least one syringe includes a pair of syringes.

### Example 24

The apparatus of any of Examples 21 through 23, further comprising a cradle configured to hold the at least one syringe, wherein the cradle is coupled to the wearable component.

### Example 25

The apparatus of Example 24, wherein the cradle is removably coupled to the wearable component.

### Example 26

The apparatus of Example 25, wherein the cradle is removably coupled to the wearable component via magnetic attraction.

### Example 27

The apparatus of any of Examples 24 through 26, wherein the cradle includes an illumination chamber configured to receive an illuminating portion of a chandelier for illuminating the at least one syringe.

### Example 28

The apparatus of Example 27, further comprising a chandelier socket configured to selectively receive the chandelier.

### Example 29

The apparatus of any of Examples 27 through 28, wherein the cradle includes a bore configured to receive the at least one syringe, wherein the illumination chamber opens into the bore via an aperture.

### Example 30

The apparatus of any of Examples 27 through 29, further comprising the chandelier, wherein the chandelier includes the illuminating portion, wherein the illuminating portion is disposed within the illumination chamber of the cradle.

### Example 31

An apparatus, comprising: (a) a first syringe including: (i) a first proximal end, (ii) a first distal end configured to dispense a first fluid, and (iii) a first piston disposed between the first proximal and distal ends; (b) a second syringe including: (i) a second proximal end, (ii) a second distal end configured to dispense a second fluid, and (iii) a second piston disposed between the second proximal and distal ends; (c) a pressurized fluid medium source; (d) an inlet port fluidly coupled with the pressurized fluid medium source; (e) a first outlet port fluidly coupled with the first proximal end of the first syringe; (f) a second outlet port fluidly coupled with the second proximal end of the second syringe; and (g) a fluid selector movable between: (i) a closed state in which the inlet port is fluidly isolated from each of the first and second outlet ports, (ii) a first open state in which the inlet port is fluidly isolated from the second outlet port and in which the inlet port is fluidly coupled with the first outlet port for directing pressurized fluid medium from the pressurized fluid medium source to the first proximal end of the first syringe to advance the first piston distally and thereby dispense the first fluid from the first distal end, and (iii) a second open state in which the inlet port is fluidly isolated from the first outlet port and in which the inlet port is fluidly coupled with the second outlet port for directing pressurized fluid medium from the pressurized fluid medium source to the second proximal end of the second syringe to advance the second piston distally and thereby dispense the second fluid from the second distal end.

### Example 32

The apparatus of Example 31, wherein the first fluid includes a biologically inert bleb fluid, wherein the second fluid includes a biologically active therapeutic fluid.

### Example 33

An apparatus, comprising: (a) a body; (b) an outer cannula extending distally from the body; (c) an inner cannula slidably disposed in the outer cannula for translating longitudinally relative to the outer cannula, wherein the inner cannula includes: (i) an open proximal end, (ii) an open distal end configured to pierce a layer of an eye of a patient to access a subretinal region of the eye, and (iii) a lumen extending between the open proximal end and the open distal end, wherein the lumen is configured to communicate first and second fluids from the open proximal end to the open distal end for delivering the first and second fluids into the subretinal region of the eye; and (d) a fluid supply assembly including: (i) first and second proximal fluid supply passageways configured to be selectively fluidly coupled to the lumen of the inner cannula for supplying the first and second fluids, respectively, to the lumen, and (ii) first and second Belleville valves configured to selectively fluidly couple the first and second proximal fluid supply passageways, respectively, to the lumen of the inner cannula.

### Example 34

The apparatus of Example 33, wherein the first and second Belleville valves are each configured to fluidly couple the respective first or second proximal fluid supply passageway to the lumen of the inner cannula in response to a fluid pressure within the respective first or second fluid passageway being equal to or greater than a threshold fluid pressure.

### Example 35

The apparatus of any of Examples 33 through 34, wherein the first and second Belleville valves are each configured to fluidly isolate the respective first or second proximal fluid supply passageway from the lumen of the inner cannula in response to a fluid pressure within the respective first or second fluid passageway being less than the threshold fluid pressure.

### V. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus, comprising:
(a) a body;
(b) an outer cannula extending distally from the body along a longitudinal axis, wherein the outer cannula is fixedly secured to the body, wherein the outer cannula includes:
(i) a proximal portion having a first cross-dimension, and
(ii) a distal portion having at least one second cross-dimension smaller than the first cross-dimension, wherein the distal portion is configured to guide the cylindrical proximal portion into a trocar port for advancing the outer cannula into an eye of a patient;
(c) an inner cannula extending along the longitudinal axis and slidably disposed in the outer cannula for translating longitudinally relative to the outer cannula, wherein the inner cannula includes:
(i) an open proximal end,
(ii) an open distal end configured to pierce a retina of the eye, and
(iii) a lumen extending between the open proximal end and the open distal end, wherein the lumen is configured to communicate at least one fluid from the open proximal end to the open distal end for delivering the at least one fluid into a subretinal region of the eye.

2. The apparatus of claim 1, wherein the distal portion tapers radially inwardly in a distal direction.

3. The apparatus of claim 1, wherein the distal portion curves radially inwardly in a distal direction.

4. The apparatus of claim 1, wherein the proximal portion is generally cylindrical.

5. The apparatus of claim 4, wherein the distal portion is generally frustoconical.

6. The apparatus of claim 1, wherein the proximal portion defines a proximal opening, wherein the distal portion defines a distal opening smaller than the proximal opening.

7. The apparatus of claim 6, wherein the distal opening is configured to maintain centration of the inner cannula relative to the outer cannula during longitudinal translation of the inner cannula relative to the outer cannula.

8. The apparatus of claim 1, wherein the open distal end is beveled.

9. The apparatus of claim 8, wherein the open distal end is oriented relative to the longitudinal axis at an angle of about 45 degrees.

10. The apparatus of claim 1, wherein the outer cannula is rigid.

11. The apparatus of claim 1, wherein the outer cannula comprises a metallic material.

12. The apparatus of claim 11, wherein the outer cannula comprises surgical stainless steel.

13. The apparatus of claim 1, wherein the inner cannula is flexible.

14. The apparatus of claim 1, wherein the inner cannula comprises a polymeric material.

15. The apparatus of claim 14, wherein the inner cannula comprises polyimide.

16. The apparatus of claim 1, the at least one fluid including first and second fluids, the apparatus further comprising first and second proximal fluid supply passageways configured to be selectively fluidly coupled to the lumen of the inner cannula for supplying the first and second fluids, respectively, to the lumen.

17. The apparatus of claim 16, further comprising first and second valves configured to selectively fluidly couple the first and second proximal fluid supply passageways, respectively, to the lumen of the inner cannula.

18. The apparatus of claim 17, wherein the first and second valves include first and second Belleville valves, respectively.

19. The apparatus of claim 17, wherein the first and second valves are each configured to fluidly couple the respective first or second proximal fluid supply passageway to the lumen of the inner cannula in response to a fluid pressure within the respective first or second fluid passageway being equal to or greater than a threshold fluid pressure.

20. The apparatus of claim 19, wherein the first and second valves are each configured to fluidly isolate the respective first or second proximal fluid supply passageway from the lumen of the inner cannula in response to a fluid pressure within the respective first or second fluid passageway being less than the threshold fluid pressure.

21-32. (canceled)

33. An apparatus, comprising:
(a) a body;
(b) an outer cannula extending distally from the body;
(c) an inner cannula slidably disposed in the outer cannula for translating longitudinally relative to the outer cannula, wherein the inner cannula includes:
(i) an open proximal end,
(ii) an open distal end configured to pierce a layer of an eye of a patient to access a subretinal region of the eye, and
(iii) a lumen extending between the open proximal end and the open distal end, wherein the lumen is configured to communicate first and second fluids from the open proximal end to the open distal end for delivering the first and second fluids into the subretinal region of the eye; and
(d) a fluid supply assembly including:
(i) first and second proximal fluid supply passageways configured to be selectively fluidly coupled to the lumen of the inner cannula for supplying the first and second fluids, respectively, to the lumen, and
(ii) first and second Belleville valves configured to selectively fluidly couple the first and second proximal fluid supply passageways, respectively, to the lumen of the inner cannula.

34. The apparatus of claim 33, wherein the first and second Belleville valves are each configured to fluidly couple the respective first or second proximal fluid supply passageway to the lumen of the inner cannula in response to a fluid pressure within the respective first or second fluid passageway being equal to or greater than a threshold fluid pressure, wherein the first and second Belleville valves are each configured to fluidly isolate the respective first or second proximal fluid supply passageway from the lumen of the inner cannula in response to a fluid pressure within the respective first or second fluid passageway being less than the threshold fluid pressure.

35. (canceled)
